(19) United States
(12) Patent Application Publication
(10) Pub. No.: US 2024/0000810 A1
(43) Pub. Date: Jan. 4, 2024

(54) USE OF CANNABINOIDS IN THE TREATMENT OF TOURETTE SYNDROME AND TIC DISORDERS

(71) Applicant: Orcosa Inc., Ewing, NJ (US)

(72) Inventors: Mark RIDALL, Skillman, NJ (US); Vincent T. MILETO, Flemington, NJ (US)

(73) Assignee: Orcosa Inc., Ewing, NJ (US)

(21) Appl. No.: 18/317,977

(22) Filed: May 16, 2023

Related U.S. Application Data
(60) Provisional application No. 63/342,925, filed on May 17, 2022.

(51) Int. Cl. A61K 31/00; A61K 47/42; A61K 47/26; A61P 25/08; A61P 25/16; A61K 9/00

(52) U.S. Cl. CPC A61K 31/658; A61K 47/42; A61K 9/006; A61P 25/08; A61P 25/16; A61K 47/26

(57) ABSTRACT

A method of treating Tourette syndrome or a tic disorder in a subject, whereby the subject in need thereof is administered, via the oral mucosa, a bioefficient rapidly infusing composition that includes (a) a pharmaceutically acceptable binder and/or excipient system containing gelatin and a sugar alcohol, and (b) a therapeutically effective amount of cannabidiol (CBD) or a derivative/analog thereof.

### CROSS-REFERENCE TO RELATED APPLICATION

The present application claims the benefit of priority to U.S. Provisional Application No. 63/342,925, filed May 17, 2022, which is incorporated herein by reference in its entirety.

### BACKGROUND OF THE INVENTION

### Technical Field

The present disclosure relates to methods of treating Tourette syndrome or a tic disorder with a rapidly infusing composition formulated with cannabidiol (CBD), cannabinoids, or a derivative/analog thereof as the active therapeutic ingredient (ATI).

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Tourette syndrome is a neurological disorder characterized by repetitive, stereotyped, abnormal involuntary movements and vocalizations called tics. Tics may also manifest in tic disorders not diagnosed as Tourette syndrome. For many patients, Tourette syndrome and tic disorders are a chronic, lifetime disease. Simple tics, for example, excessive eye blinking or brief facial grimacing, are relatively easy to hide and may go largely unnoticed by others. Complex tics, such as body contortions, self-injurious behavior, obscene gestures, or shouting of socially inappropriate word or phrases, may appear intentional rather than involuntary. Such complex tics can be particularly distressing and embarrassing to a person suffering from Tourete syndrome or a tic disorder given the attention the behaviors can draw from others. Even if those complex tics go unnoticed, they can be distracting and disabling in a person's normal course of functioning. However, even less severe tics such as excessive blinking can negatively interfere with a patient's quality of life.

In some individuals, Tourette syndrome symptoms improve in adolescence and the number and/or severity of tics decline (or even cease entirely) in adulthood. However, in some individuals, the tics persist even in adulthood, and may even increase in number and severity, thus requiring long-term treatment. It is estimated that I0%-15% of patients with Tourette syndrome have a disabling disease course that lasts into adulthood.

In addition to tics, patients with Tourette syndrome often experience other neurobehavioral symptoms such as attention deficit hyperactivity disorder (ADHD), difficulties with reading and concentrating, obsessive-compulsive thoughts, and repetitive behaviors. It has been estimated that 90% of patients with Tourette syndrome suffer from comorbid neuropsychiatric disorders, most commonly ADHD and obsessive-compulsive disorder (OCD).

There is currently no medication that is helpful to all patients with Tourette syndrome. While neuroleptic drugs (i.e., antipsychotics) have been effective for treatment of tics in a few patients, these medications do not entirely eliminate tic symptoms and have significant side effects. In addition, treatment of neurobehavioral disorders associated with Tourette syndrome, such as ADHD, may be complicated as some medications used to treat ADHD are contraindicated in patients with Tourette syndrome. Therefore, new treatments with minimal side effects and general efficacy are needed to treat the spectrum of symptoms of Tourette syndrome and tic disorders.

The administration of CBD has been suggested as a treatment for Tourette syndrome among several other medical conditions. See U.S. Patent Publication 20030017216A 1-incorporated herein by reference in its entirety. Epidiolex® is currently the only FDA-approved drug containing CBD as the active ingredient. With this particular drug the CBD is formulated in an oral liquid dosage form with inactive ingredients dehydrated alcohol, sesame oil, strawberry flavor, and sucralose for the treatment of epilepsy, specifically for the treatment of seizures associated with Lennox-Gastaut syndrome, Dravet syndrome, or Tuberous Sclerosis Complex.

However, the oral liquid dosage administration of Epidiolex® suffers from a number of disadvantages. For example, treatment with Epidiolex® typically requires extremely high doses of CBD, with the FDA-approved recommended dosages of CBD being in the range of 5 mg/kg/day to 25 mg/kg/day for the treatment of epilepsy, divided between two daily doses. These extremely high doses are an unavoidable consequence of oral liquid administration, which results in low bioavailability and inconsistent levels of CBD in systemic circulation. Specifically, drugs taken by mouth and swallowed are absorbed first into the blood perfusing the gastrointestinal (GI) tract. The venous drainage from the G1 tract is into the blood perfusing the liver, and thus drugs absorbed from the lumen of the GI tract are immediately presented to the liver—the major detoxifying organ of the body—whereby the drugs are metabolized and then returned to the left side of the heart via the hepatic portal vein and sent into systemic circulation. This first pass metabolism through the liver may result in the removal of a substantial portion of an ingested drug and is more pronounced for some drugs than others; in the case of cannabinoids such as CBD, extensive first pass metabolism provides a paltry bioavailability of only about 6 to 11% when ingested orally. This bioavailability is further affected by whether the subject is in a “fasted” or a “fed” state and even the content of the meal for subjects in the “fed” state.

Another disadvantage associated with medications administered thorough oral liquid dosing is inaccurate dosing. To use Epidiolex®, subjects are instructed to ill a 5 mL oral syringe with the required dosage of the oral solution and then to squirt the contents into the mouth. It is recommended to direct the medication against the inside of the cheek because directing the medication towards the back of the mouth may cause choking. Since each mL of Epidiolex® contains 100 mg of CBD, the instructions recognize that many patients will require greater than 5 mL of oral solution during each of the two daily dosing events to reach the recommended dosage amount, requiring multiple loadings and dispensings of the oral syringe. Any inaccuracy in each of the multiple, daily oral syringe loadings and dispensings can result in large variations in the amount of CBD dosed. This process is not only inaccurate, it is cumbersome and generally unpleasant for the patient. Additionally, the oily and foul taste of CBD exacerbates the unpleasant user experience and can result in poor patient compliance when administered orally.

Given the inconsistencies in bioavailability and dosing of CBD using Epidiolex®, it is also difficult to reliably increase or decrease the amount of CBD in systemic circulation in response to patient outcomes. For instance, given the wide range of Tourette syndrome symptoms and their tendency to change over the course of a patient's life, especially starting at a young age, a patient may respond better to greater or smaller amounts of CBD. However, for the reasons discussed above, a dosage increase of 20% for CBD in an oral liquid dosage form, for example, would not dependably cause a 20% increase of CBD in systemic circulation.

Yet another disadvantage of the oral liquid dosage administration of Epidiolex® is the adverse reactions associated with consuming voluminous amounts of the liquid carrier, specifically sesame oil, needed to meet the high daily dosing requirements of CBD (5 mg/kg/day to 25 mg/kg/day). As a result, taking Epidiolex® is known to cause gastrointestinal ailments such as diarrhea, decreased weight, gastroenteritis, decreased appetite, and abdominal pain/discomfort. Such adverse reactions increase in patients receiving higher doses of Epidiolex® for the treatment of Tourette syndrome and tic disorders.

In view of the foregoing, there exists a need for a bioefficient Tourette syndrome and tic disorder treatment using CBD based on dosage forms which are bioavailable, are easy to administer, provide for accurate dosing, minimize adverse reactions, and result in high levels of patient compliance.

### SUMMARY OF THE INVENTION

In U.S. provisional application 63/114,194 and U.S. non-provisional application Ser. No. 17/225,738—each incorporated herein by reference in its entirety, the instant inventors described a rapidly infusing composition containing cannabidiol (CBD) as the active therapeutic ingredient (ATI) and corresponding methods for treating pain using that composition. The inventive rapidly infusing composition provides numerous benefits compared to traditional modes of dosing CBD, such as the oral liquid dosing required with Epidiolex®, including, but not limited to: higher bioavailability; more rapid uptake; more accurate dosing; greater convenience; fewer side effects; and superior patient compliance.

As described in more detail below, the Rapid Infusion Technology® (RITe) platform formulated with CBD or cannabinoids enables rapid infusion of CBD into systemic circulation via the oral mucosa while bypassing the GI tract and hepatic first pass metabolism, and provides for the first time a consistent, repeatable mechanism for the treatment of Tourette syndrome in a bioavailable unit dosage form for accurate dosing, easy administration, smaller dosages, reduced side effects, and high levels of patient compliance.

Thus, the present invention provides:
(1) A method of treating Tourette syndrome in a subject, comprising:
administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD).
(2) The method of (1), wherein the rapidly infusing composition is lyophilized.
(3) The method of (1) or (2), wherein the rapidly infusing composition has a disintegration time of approximately 1 to 30 seconds in deionized water maintained at 37° C. 2° C.
(4) The method of any one of (1) to (3), wherein the rapidly infusing composition has a disintegration time of approximately 1 to 5 seconds in deionized water maintained at 37° C. 2° C.
(5) The method of any one of (1) to (4), wherein the gelatin is present in the rapidly infusing composition in an amount of 10 to 35 wt. %, based on a total weight of the rapidly infusing composition on a dry basis.
(6) The method of any one of (1) to (5), wherein the gelatin is bovine gelatin.
(7) The method of any one of (1) to (6), wherein the sugar alcohol is present in the rapidly infusing composition in an amount of 5 to 35 wt. %, based on a total weight of the rapidly infusing composition on a dry basis.
(8) The method of any one of (1) to (7), wherein the sugar alcohol comprises mannitol.
(9) The method of any one of (1) to (8), wherein the CBD is present in the rapidly infusing composition in an amount of 20 to 70 wt. %, based on a total weight of the rapidly infusing composition on a dry basis.
(10) The method of any one of (1) to (9), wherein the rapidly infusing composition is formulated with a solid form of the CBD.
(11) The method of any one of (1) to (10), wherein the rapidly infusing composition is formulated with a solid form of the CBD having a purity between 95 and 99.9 wt. %.
(12) The method of any one of (1) to (11), wherein the rapidly infusing composition is formulated with a solid form of the CBD that has been micronized to have a DSO diameter between 1 and 50 μm.
(13) The method of any one of (1) to (12), wherein the rapidly infusing composition further comprises at least one selected from the group consisting of a sweetener, a flavorant, and a colorant.
(14) The method of (13), wherein the rapidly infusing composition comprises the flavorant, and the flavorant comprises lemon-lime flavor.
(15) The method of (13) or (14), wherein the rapidly infusing composition comprises the colorant, and the colorant comprises FD&C Yellow #5.
(16) The method of any one of (13) to (15), wherein the rapidly infusing composition comprises the sweetener, and the sweetener comprises a mixture of sucralose and acesulfame-K.
(17) The method of any one of (1) to (16), wherein the rapidly infusing composition is administered to the subject via the buccal mucosa.
(18) The method of any one of (1) to (17), wherein the therapeutically effective amount of CBD is from 0.1 mg/kg/day to less than 7 mg/kg/day.
(19) The method of any one of (1) to (17), wherein the therapeutically effective amount of CBD is from 0.1 mg/kg/day to less than 5 mg/kg/day.
(20) The method of any one of (1) to (19), wherein the rapidly infusing composition is administered to the subject 1 to 3 times per day.
(21) The method of any one of (1) to (20), wherein CBD is the only active therapeutic ingredient in the rapidly infusing composition.
(22) The method of any one of (1) to (21) wherein the subject is not administered a cannabinoid other than CBD.
(23) The method of any one of (1) to (22), wherein the subject presents with at least one symptom of an abnormal involuntary movement selected from the group consisting of an eye movement, a facial movement, a shoulder movement, a head movement, a respiratory movement, and a vocalization.
(24) The method of any one of (1) to (23), wherein the subject presents with a complex symptom.
(25) The method of (24), wherein the complex symptom is at least one speech-related symptom selected from the group consisting of coprolalia, echolalia, and palilalia.
(26) The method of (24), wherein the complex symptom is a motor-related symptom involving several muscle groups.
(27) The method of any one of (24) or (26), wherein the complex symptom is at least one selected from the group consisting of hopping, jumping, bending, twisting, brushing hair, throwing objects, touching objects, copropraxia, and echopraxia.
(28) The method of (23), wherein the subject presents with at least two symptoms selected from the group, and
wherein the at least two symptoms are exhibited simultaneously as a complex symptom.
(29) The method of any one of (1) to (28), wherein the subject presents with at least one condition selected from the group consisting of stuttering, attention deficit hyperactivity disorder, obsessive-compulsive disorder, and autism spectrum disorder.
(30) The method of any one of (1) to (29), wherein the subject is administered the rapidly infusing composition in the absence of an environmental modification comprising a trigger.
(31) The method of any one of (1) to (30), wherein a tic severity or frequency in the subject is reduced by at least 10%, relative to a severity or frequency observed prior to administration of the rapidly infusing composition.
(32) The method of any one of (1) to (31), wherein a tic severity or frequency in the subject is reduced by at least 20%, relative to a severity or frequency observed prior to administration of the rapidly infusing composition.
(33) The method of any one of (1) to (32), wherein a tic severity or frequency in the subject is reduced by at least 40%, relative to a severity or frequency observed prior to administration of the rapidly infusing composition.
(34) The method of any one of (1) to (33), wherein a premonitory urge for tics of the subject as measured by a Premonitory Urge for Tics Scale (PUTS) is reduced by at least 20%, relative to a premonitory urge for tics prior to administration of the rapidly infusing composition.
(35) The method of any one of (1) to (34), wherein a tic severity of the subject as measured by the Yale Global Tic Severity Scale is reduced by at least 10%, relative to a tic severity prior to administration of the rapidly infusing composition.
(36) The method of any one of (1) to (35), wherein a tic severity of the subject as measured by the Yale Global Tic Severity Scale is reduced by at least 25%, relative to a tic severity prior to administration of the rapidly infusing composition.
(37) The method of any one of (1) to (36), wherein a tic severity of the subject as measured by the Yale Global Tic Severity Scale is reduced by at least 50%, relative to a tic severity prior to administration of the rapidly infusing composition.
(38) The method of any one of (1) to (37), wherein the occurrence of tics in the subject is reduced completely.
(39) The method of any one of (1) to (38), wherein the subject is additionally treated with deep brain stimulation.
(40) The method of any one of (1) to (39), wherein the rapidly infusing composition is administered in combination with at least one drug selected from the group consisting of a dopamine precursor, a DOPA decarboxylase inhibitor, a catechol-O-methyl transferase (COMT) inhibitors, a dopamine receptor agonist, a neuroprotective agent, an NMDA antagonist, an anti-psychotic, a benzodiazepine, and a CYP2D6 inhibitor.
(41) The method of any one of (1) to (40), wherein the rapidly infusing composition is administered in combination with at least one anti-psychotic drug selected from the group consisting of chlorpromazine, levomepromazine, promazine, acepromazine, trinflupromazine, cyamemazine, chlorproethazine, dixyrazine, fluphenazine, perphenazine, prochlorperazine, thiopropazate, trifluoperazine, acetophenazine, thioproperazine, butaperazine, perazine, periciazine, thioridazine, mesoridazine, pipotiazine, haloperidol, fluphenazine, trifluperidol, melperone, moperone, pipamperone, bromperidol, benperidol, droperidol, fluanisone, oxypertine, molindone, sertindole, ziprasidone, flupentixol, clopenthixol, chlorprothixene, thiothixene, zuclopenthixol, fluspirilene, pimozide, penfluridol, loxapine, clozapine, olanzapine, quetiapine, tetrabenazine, sulpiride, sultopride, tiapride, remoxipride, amisulpride, veralipride, levosulpiride, lithium, prothipendyl, risperidone, clotiapine, mosapramine, zotepine, pripiprazole, and paliperidone.
(42) The method of any one of (1) to (41), wherein the rapidly infusing composition is administered in combination with at least one benzodiazepine selected from the group consisting of alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.
(43) The method of any one of (1) to (42), wherein the rapidly infusing composition is administered in combination with at least one CYP2D6 inhibitor selected from the group consisting of fluoxetine, paroxetine, bupropion, quinidine, cinacalcet, and ritonavir.
(44) A method for the treatment of a tic disorder in a subject, the method comprising: administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD),
wherein the subject does not have Tourette syndrome.
(45) The method of (44), wherein the subject has at least one tic disorder selected from the group consisting of a pediatric autoimmune disorder associated with streptococcal infection (PANDAS), a transient tic disorder, and a chronic tic disorder.
(46) The method of (44) or (45), wherein the subject has a movement disorder.

### DETAILED DESCRIPTION OF THE INVENTION

In the following description, it is understood that other embodiments may be utilized and structural and operational changes may be made without departure from the scope of the present embodiments disclosed herein.

### Definitions

As used herein, the terms “compound” and “product” are used interchangeably, and are intended to refer to a chemical entity, whether in the solid, liquid or gaseous phase, and whether in a crude mixture or purified and isolated. Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Many geometric isomers of C═C double bonds, C═N double bonds, ring systems, and the like can also be present, and all such stable isomers are contemplated in the present disclosure. Cis- and trans- (or E- and Z-) geometric isomers, when present, may be isolated as a mixture of isomers or as separated isomeric forms. Compounds referenced in the disclosure can be isolated in optically active or racemic forms. Optically active forms may be prepared by resolution of racemic forms or by synthesis from optically active starting materials. All processes used to prepare these compounds and intermediates made therein are considered to be part of the present disclosure. When enantiomeric or diastereomeric products are prepared, they may be separated by conventional methods, for example, by chromatography, fractional crystallization, or through the use of a chiral agent. Depending on the process conditions, the end products referenced in the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds may be separated into the individual isomers. Compounds referenced in the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure. Further, a given chemical formula or name shall encompass all conformers, rotamers, or conformational isomers thereof where such isomers exist. Different conformations can have different energies, can usually interconvert, and are very rarely isolatable. There are some molecules that can be isolated in several conformations. For example, atropisomers are isomers resulting from hindered rotation about single bonds where the steric strain barrier to rotation is high enough to allow for the isolation of the conformers. It should be understood that all conformers, rotamers, or conformational isomer forms, insofar as they may exist, are included within the present disclosure.

As used herein, the term “solvate” refers to a physical association of a referenced compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvent molecules which may form the solvate include, but are not limited to, water, methanol, ethanol, n-propanol, isopropanol, n-butanol, isobutanol, tert-butanol, ethyl acetate and other lower alkanols, glycerin, acetone, dichloromethane (DCM), dimethyl sulfoxide (DMSO), dimethyl acetate (DMA), dimethylformamide (DMF), isopropyl ether, acetonitrile, toluene, N-methylpyrrolidone (NMP), tetrahydrofuran (THF), tetrahydropyran, other cyclic mono-, di- and tri-ethers, polyalkylene glycols (e.g., polyethylene glycol, polypropylene glycol, propylene glycol), and mixtures thereof in suitable proportions. Exemplary solvates include, but are not limited to, hydrates, ethanolates, methanolates, isopropanolates and mixtures thereof. Methods of solvation are generally known to those of ordinary skill in the art.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids and phenols. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990)—which is incorporated herein by reference in its entirety.

When referencing a particular composition/material, the phrase “consists essentially of”, means that the particular composition/material may include minor amounts of impurities so long as those impurities do not affect the basic and novel property of the invention—the ability to treat Tourette syndrome and tic disorders.

As used herein, the terms “optional” or “optionally” means that the subsequently described event(s) can or cannot occur or the subsequently described component(s) may or may not be present (e.g., 0 wt. %).

As used herein, the terms “treat”. “treatment”, and “treating” in the context of the administration of a therapy to a subject in need thereof refers to the reduction or amelioration of severity of symptoms of the condition being treated; reduction of duration of symptoms of the condition being treated; reduction, inhibition, slowing, or arresting of the progression of symptoms associated with the condition; reduction of frequency of symptoms of the condition being treated; elimination of symptoms and/or underlying cause of the condition;

prevention of the occurrence of symptoms of the condition, for example in a subject that may be predisposed to the condition but does not yet experience or exhibit symptoms of the condition; improvement or remediation or amelioration of damage following a condition, for example improving, remediating, or ameliorating neurological damage or other physical damage associated with Tourette syndrome and tic disorders; providing a prophylactic or preventative measure against an abnormal involuntary movement; reducing a premonitory urge; and/or causing regression of the condition.

The term “prophylactic” or “preventative” when used refers to the administration of an active compound to prevent, reduce the likelihood of an occurrence or a reoccurrence of an abnormal involuntary movement that would occur without such treatment.

The term “subject” and “patient” are used interchangeably. As used herein, they refer to any subject for whom or which therapy is desired. In most embodiments, the subject is a human.

The terms “administer” “administering”, “administration”, and the like, as used herein, refer to the methods that may be used to enable delivery of the active therapeutic ingredient (ATI) to the desired site of biological action. Routes or modes of administration are as set forth herein.

The term “Rapid Infusion Technology™ (RITe) platform” or “rapidly infusing composition”, as used herein means a solid dosage form containing medicinal substances that disintegrates rapidly in the oral cavity (when contacted with saliva) with no need for chewing or drinking/swallowing liquids (e.g., water, liquid carriers, saliva, etc.) to ingest these medicinal substances, with an in-vitro disintegration time of 30 second or less according to the United States Pharmacopeia (USP)<701> Disintegration Test. The disclosed rapidly infusing compositions are thus a different dosage form than, for example, a chewable tablet, a lozenge intended to be dissolved slowly in the mouth, an orally disintegrating film or tablet designed to be dissolved/disintegrated in the mouth and swallowed (also called “orodispersible” formulations), a tablet that should be swallowed whole with food or liquid, oral liquid dosage forms, or any other oral dosage form designed for absorption from the GI tract.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. The terms “effective amount” or “therapeutically effective amount” refer to a sufficient amount of an active therapeutic ingredient (ATI) being administered which provides the desired therapeutic or physiological effect or outcome, for example, the amount of ATI sufficient for reducing the number, frequency, intensity, complexity, and/or interference of tics or abnormal involuntary movements. The result can be a reduction and/or alleviation of the signs, symptoms, or causes of a disease, or any other desired alteration of a biological system. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the age and general condition of the subject, mode of administration, and the like. An appropriate “effective amount” in any individual case may be determined by one of ordinary skill in the art using only routine experimentation, for example through the use of dose escalation studies.

### Rapid Infusion Technology™ (RITe) Platform

The present disclosure provides a therapeutic formulation presented in the form of a rapidly infusing composition which is suitable for administration of lipophilic active therapeutic ingredients (ATIs) such as cannabidiol (CBD) via a non-gastric mucosal surface. As described in more detail below, the novel delivery platform allows otherwise difficult to formulate ATIs—such as CBD—to be presented in unit dosage form for accurate dosing and in an easy-to-take format for high levels of patient compliance. For example, the rapidly infusing composition may be presented in tablet form and packaged in individual blister units.

In particular, the rapidly infusing composition enables oral mucosal administration of lipophilic ATIs in a solid dosage form directly into systemic circulation via the sublingual mucosa or the buccal mucosa and avoidance of first pass metabolism. The rapidly infusing composition thus presents lipophilic ATIs such as CBD (which may otherwise be susceptible to extensive first pass metabolism) in an enhanced bioefficient dosage form. Such exemplary dosage forms may include, inter alia, enhanced bioavailability, typically with a bioavailability of at least 20%, preferably at least 30%, preferably at least 40%, preferably at least 50%, preferably at least 60%, preferably at least 70%, preferably at least 80%, preferably at least 90%, and up to 95%, preferably up to 90%, preferably up to 80%, preferably up to 70%, preferably up to 60%, preferably up to 50%, preferably up to 40%, preferably up to 30%. Such enhanced bioavailability allows the dosage amount of ATI to be reduced, whilst maintaining the same pharmacological effect. For example, the RITe™ platform allows CBD to be dosed in an amount of 7 mg/kg/day, preferably under 5 mg/kg/day for the treatment of Tourette syndrome and/or a tic disorder.

Additionally, the bioefficient rapidly infusing composition enables a defined dose of ATI to be absorbed via the oral mucosae, prior to the gastric mucosa, thereby presenting a defined and consistent level of ATI into systemic circulation for consistent and reliable pharmacological effects. Consistency in pharmacological effects helps to improve patient adherence during treatment. The aforementioned enhanced levels of bioavailability may be consistently achieved because the RITe™ platform reduces the tendency for enteral oral administration through voluntary or involuntary swallowing by shortening the residence time the ATI spends in the oral cavity. Any amount of ATI (e.g., CBD) that is swallowed would be subject to first-pass metabolism and thus overall lower bioavailability. Swallowing further results in greater variability in the effective amount of dosing, as a result of variability in the amount swallowed and the greater subject variability of bioavailability through first-pass metabolism for the amount swallowed.

Administration may be carried out by simply placing the rapidly infusing composition directly in the buccal cavity (between the cheek and gum) or over the sublingual mucous gland (under the ventral surface of the tongue). Preferred rapidly infusing compositions are those which are lyophilized products formulated for rapid disintegration when placed in such an oral environment for rapid release of the ATI. The rapidly infusing compositions of the present disclosure may have a disintegration time of from approximately 1 second to 30 seconds or less, preferably 25 seconds or less, preferably 20 seconds or less, preferably 15 seconds or less, preferably 10 seconds or less, preferably 5 seconds or less, preferably 3 seconds or less, according to the United States Pharmacopeia (USP)<701> Disintegration Test performed in deionized water maintained at 37° C.±2°. In particular, preferred rapidly infusing compositions are those formulated for oral disintegration in 5 seconds or less, preferably 4 seconds or less, preferably 3 seconds or less, preferably 2 seconds or less, preferably in approximately 1 second, according to the United States Pharmacopeia (USP)<701> Disintegration Test performed in deionized water maintained at 37° C.±2°. A disintegration profile no higher than the above-mentioned upper limit when in intimate contact with a non-gastric mucosal surface provides for rapid absorption of the ATI and short onset times to therapeutic relief.

The easy-to-take oral mucosal dosage form presented herein is a vast improvement over the cumbersome and generally unpleasant oral liquid dosage form of Epidiolex®, which as discussed heretofore, is known to cause gastrointestinal side effects and often requires multiple loadings and dispensings of the oral syringe to meet the prescribed high dosages—a particular concern if administering to children. In contrast, administration of the rapidly infusing composition of the present disclosure is easy, one simply “takes it and it's gone,” with no need for swallowing, thus offering improved patient compliance whether self-administered or administered by a healthcare provider, a care-taker, etc.

Patient compliance may also be improved in terms of temporary abstinence from swallowing, which is often triggered when a patient is presented with foul-tasting oral medications. Any issues related to foul taste may be minimized with the above rapid disintegration times, which reduces the tendency for enteral oral administration through voluntary or involuntary swallowing, and as a result, the aforementioned enhanced levels of bioavailability may be achieved.

The rapid disintegration profile disclosed herein, coupled with the direct introduction of the ATI into systemic circulation through the sublingual mucosa or the buccal mucosa, preferably through the buccal mucosa, provides a more rapid onset of therapeutic effect. For example, the rapidly infusing composition may provide the desired relief from Tourette syndrome or a tic disorder symptom, including premonitory urge, in (has an onset time of) under 15 minutes, preferably under 10 minutes, preferably under 5 minutes, preferably under 4 minutes, preferably under 3 minutes, preferably under 2 minutes, preferably under 1 minute, preferably under 45 seconds, preferably under 30 seconds, preferably under 20 seconds, preferably under 10 seconds, preferably approximately 5 seconds. Such short onset times are superior to those which can be obtained with traditional oral dosage forms such as tablets taken with food or liquids, liquid dosage forms, as well as orodispersible dosage forms dissolved by mouth and then swallowed. Relief from some symptoms of Tourette syndrome or tic disorders may have a longer onset time period, for instance, under 5 days, preferably under 3 days, preferably under 1 day.

The rapidly infusing composition herein generally contains (a) a pharmaceutically acceptable binder and/or excipient system that includes gelatin and a sugar alcohol e.g., mannitol, and optionally one or more of a sweetener, a flavorant, and a colorant; and (b) a therapeutically effective amount of an active therapeutic ingredient such as cannabidiol (CBD).

Pharmaceutically Acceptable Carrier and/or Excipient System

Carriers and/or excipients are ingredients which do not provide a therapeutic effect themselves, but which are designed to interact with, and enhance the properties of, the active therapeutic ingredient. In particular, carriers and/or excipients may act as a vehicle for transporting the active therapeutic ingredient from one organ, or portion of the body, to another organ, or portion of the body. The selection of appropriate carrier/excipient ingredients may impact the solubility, distribution, release profile/kinetics, absorption, serum stability, therapeutic onset time, and ultimately the efficacy of the ATI, as well as the shelf-life, dosage forms, and processability of the drug product. Each ingredient in the pharmaceutically acceptable carrier and/or excipient system must be “pharmaceutically acceptable” in the sense of being compatible with the other ingredients of the rapidly infusing composition and not injurious to the patient.

In light of the above, particular preference is given herein to pharmaceutically acceptable carrier and/or excipient systems which include gelatin and a sugar alcohol (e.g., mannitol).

Gelatin is to be included in the pharmaceutically acceptable carrier and/or excipient system in order to effect matrix formation in the lyophilized product, i.e., gelatin may act primarily as a matrix former. During manufacture of the rapidly infusing composition, lyophilization from an aqueous suspension results in the removal of water thereby leaving behind a gelatin matrix/scaffolding/lattice upon which the ATI can be evenly dispersed or suspended. It has been found that gelatin has a propensity to establish a stable matrix in lyophilized form, yet allow for rapid disintegration when brought into contact with the aqueous oral environment, thereby providing efficient transfer of the ATI from the hydrophilic vehicle to the oral mucosa. In this regard, mammalian gelatins such as bovine gelatin and porcine gelatin are preferred, with bovine gelatin being particularly preferred. In some embodiments, the rapidly infusing composition does not contain fish gelatin.

The amount of gelatin used may be varied. Generally, gelatin may be present in the rapidly infusing composition in an amount of at least 10 wt. %, preferably at least 12 wt. %, preferably at least 14 wt. %, preferably at least 16 wt. %, preferably at least 18 wt. %, preferably at least 20 wt. %, preferably at least 22 wt. %, and up to 50 wt. %, preferably up to 45 wt. %, preferably up to 40 wt. %, preferably up to 35 wt. %, preferably up to 32 wt. %, preferably up to 30 wt. %, preferably up to 28 wt. %, preferably up to 26 wt. %, preferably up to 24 wt. %, based on a total weight of the rapidly infusing composition on a dry basis.

The pharmaceutically acceptable carrier and/or excipient system is also formulated with one or more sugar alcohols, which may act primarily as a bulking agent. Examples of sugar alcohols include, but are not limited to, erythritol, xylitol, sorbitol, maltitol, mannitol, lactitol, and glycerin, which may be used singly or in combinations. Advantage can also be taken of the effect of certain sugar alcohols in terms of taste (sweetness and coolness due to endothermal beat of solution), as well as their ability to aid/speed tablet disintegration. In this regard, particular preference is given to mannitol.

The sugar alcohol, preferably mannitol, may be present in the rapidly infusing composition in any amount which provides the desired bulking/taste/disintegration effects. Generally, this amount will range from of at least 5 wt. %, preferably at least 10 wt. %, preferably at least 12 wt. %, preferably at least 14 wt. %, preferably at least 16 wt. %, preferably at least 18 wt. %, and up to 50 wt. %, preferably up to 45 wt. %, preferably up to 40 wt. %, preferably up to 35 wt. %, preferably up to 30 wt. %, preferably up to 28 wt. %, preferably up to 26 wt. %, preferably up to 24 wt. %, preferably up to 22 wt. %, preferably up to 20 wt. %, based on a total weight of the rapidly infusing composition on a dry basis.

In some embodiments, a weight ratio of gelatin to sugar alcohol ranges from 1:3, preferably from 1:2, preferably from 1:1, preferably from 1.1:1, and up to 3:1, preferably up to 2:1, preferably up to 1.5:1, preferably up to 1.2:1.

The pharmaceutically acceptable carrier and/or excipient system may also optionally include one or more of a sweetener, a flavorant, and a colorant.

The sweetener may be used in any amount which provides the desired sweetening effect, generally in amount of 0 to 10 wt. %, for example in an amount of up to 10 wt. %, preferably up to 8 wt. %, preferably up to 6 wt. %, preferably up to 5 wt. %, preferably up to 4 wt. %, preferably up to 3 wt. %, preferably up to 2 wt. %, preferably up to 1.5 wt. %, preferably up to 1 wt. %, preferably up to 0.5 wt. %, preferably up to 0.1 wt. %, based on a total weight of the rapidly infusing composition on a dry basis. Suitable examples of sweeteners include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, and glucose, which may be used singly or in combinations, with particular preference given to sucralose and acesulfame-K.

It is to be readily appreciated by those of ordinary skill in the art that one or more flavorants may be optionally included in the rapidly infusing composition to mask any unpleasant taste imparted by certain ingredients (e.g., an unpleasant tasting ATI) or to otherwise impart an acceptable taste profile to the composition, and the composition is not limited to any particular flavor. Suitable flavorants include, but are not limited to, oil of wintergreen, oil of peppermint, oil of spearmint, oil of sassafras, oil of clove, cinnamon, anethole, menthol, thymol, eugenol, eucalyptol, lemon, lime, lemon-lime, orange, and other such flavor compounds to add fruit notes (e.g., citrus, cherry etc.), spice notes, etc., to the composition. The flavorants may be constitutionally composed of aldehydes, ketones, esters, acids, alcohols (including both aliphatic and aromatic alcohols), as well as mixtures thereof. Specific mention is made to lemon-lime flavor powder, which works particularly well with CBD as the ATI. The flavorant may be used in any amount which provides the desired flavor, generally in an amount of 0 to 10 wt. %, for example in an amount of up to 10 wt. %, preferably up to 8 wt. %, preferably up to 6 wt. %, preferably up to 5 wt. %, preferably up to 4 wt. %, preferably up to 3 wt. %, preferably up to 2 wt. %, preferably up to 1.5 wt. %, preferably up to 1 wt. %, preferably up to 0.5 wt. %, preferably up to 0.1 wt. %, based on a total weight of the rapidly infusing composition on a dry basis.

Two main strategies contribute to the taste masking success of the present disclosure. First, any issues related to foul taste are fundamentally mitigated by the short oral residence times provided by the rapid disintegration profile described heretofore. One “takes it and it's gone.” Second, when formulated with a flavorant, a robust mixture of flavors may hit the tongue at essentially the same time—the flavor of the CBD still may hit the tongue, but the perception of the flavor is canceled or mitigated by the simultaneous arrival of other flavors. Even then, the robust mixture of flavors will quickly subside as the composition is rapidly absorbed through the oral mucosa.

Likewise, the rapidly infusing composition may be colored or tinted through the optional use of one or more colorants. Suitable colorants are those approved by appropriate regulatory bodies such as the FDA and those listed in the European Food and Pharmaceutical Directives and include both pigments and dyes such as FD&C and D&C dyes, with specific mention being made to FD&C Yellow #5.

In addition to gelatin and a sugar alcohol (e.g., mannitol), and optionally one or more of a sweetener, a flavorant, and a colorant, the pharmaceutically acceptable carrier and/or excipient system may optionally include one or more other pharmaceutically acceptable carriers and/or excipients. Examples of which include, but are not limited to,
fillers or extenders such as starches (e.g., corn starch and potato starch), sugars (e.g., lactose or milk sugar, maltose, fructose, glucose, trehalose, sucrose), dextrates, dextrin, polydextrose, high molecular weight polyethylene glycols, silicic acid, potassium sulfate, aluminum monostearate, polyesters, polycarbonates, and polyanhydrides;
binders, such as cellulose and its derivatives, (e.g., carboxymethyl cellulose, sodium carboxymethyl cellulose, hydroxypropyl cellulose, hydroxyethyl cellulose, hydroxypropylmethyl cellulose (hypromellose), hydroxyethyl methyl cellulose, methyl cellulose, ethyl cellulose, cellulose acetate, cellulose acetate phthalate, and microcrystalline cellulose), alginates (e.g., sodium alginate), polyvinyl pyrrolidone, polyvinyl acetate-vinylpyrrolidone, polyacrylic acid, methacrylate copolymers (e.g., methyl methacrylate copolymers and Eudragit® products available from Evonik), modified starch, powdered tragacanth, malt, acacia (gum arabic), carbomer/carboxyvinyl polymer, carrageenan, chitosan, copovidone, cyclodextrins and modified cyclodextrins, guar gum, inulin, pectin (e.g., low viscosity pectin), polycarbophil or a salt thereof, polyvinyl alcohol, pullulan, xanthan gum, casein, protein extracts (e.g., whey protein extract, soy protein extract), zein, levan, elsinan, gluten, locust bean gum, gellan gum, and agar;
disintegrating agents, such as agar-agar, calcium carbonate, tapioca starch, alginic acid, certain silicates, sodium carbonate, sodium starch glycolate, and cross-linked sodium carboxymethyl cellulose (croscarmellose sodium);
surfactants/absorption accelerators/wetting agents/emulsifying agents/solubilizers, including any of the anionic, cationic, nonionic, zwitterionic, amphoteric and betaine variety, such as polyalkylene oxide copolymers (e.g., poloxamers, polyethylene oxide-polypropylene oxide copolymers), sodium lauryl sulfate, sodium dodecyl benzene sulfonate, sodium docusate, sodium lauryl sulfoacetate, alkali metal or ammonium salts of lauroyl sarcosinate, myristoyl sarcosinate, palmitoyl sarcosinate, stearoyl sarcosinate and oleoyl sarcosinate, cetyl alcohol, glycerol monostearate, glycerol oleate, fatty acid mono- and di-esters of glycerol, fatty acid esters of polyethylene glycol, polyoxyethylene sorbitol, fatty acid esters of sorbitan, polysorbates (polyalkolyated fatty acid esters of sorbitan)(e.g., polyoxyethylene sorbitan monostearate, monoisostearate and monolaurate), polyethylene oxide condensates of alkyl phenols, cocoamidopropyl betaine, lauramidopropyl betaine, palmityl betaine, glyceryl monooleate, glyceryl monostearate, fatty alcohols (e.g., cetostearyl and cetyl alcohol), medium chain triglycerides, medium chain fatty acids, polyethoxylated castor oil, polyethoxylated alkyl ethers (e.g., ethoxylated isostearyl alcohols), polyethylene glycols (Macrogols), polypropylene glycols, polyoxyethylene stearates, anionic and nonionic emulsifying waxes, propylene glycol alginates, alcohol-oil transesterification products, polyglycerized fatty acids, propylene glycol fatty acid esters, mixtures of propylene glycol fatty acid esters and glycerol fatty acid esters, sterol and sterol derivatives, sugar esters, lower alcohol fatty acid esters, fatty acids and bile acids and their corresponding salts, ricinoleic acid/sodium ricinoleate, linoleic acid/sodium linoleate, lauric acid/sodium laurate, mono-, di-, and tri-hydroxy bile acids and their salts, sulfated bile salt derivatives, phospholipids, ether carboxylates, succinylated monoglycerides, mono/diacetylated tartaric acid esters of mono- and diglycerides, citric acid esters of mono- and diglycerides, alginate salts, and lactylic esters of fatty acids;
plasticizers such as glycerin fatty acid esters, sucrose fatty acid esters, lecithin (e.g., enzyme modified lecithin), polysorbates, sorbitan fatty acid esters, polyethylene glycol, propylene glycol, triacetin, glycerol oleate, medium chain fatty acids, tributyl citrate, triethyl citrate, acetyl tri-n-butyl citrate, diethyl phthalate, castor oil, dibutyl sebacate, and acetylated monoglycerides;
absorbents, such as kaolin and bentonite clay;
lubricants, such as talc, calcium stearate, magnesium stearate, solid polyethylene glycols, zinc stearate, sodium stearate, stearic acid, ethyl oleate, and ethyl laurate;
controlled release agents such as cross-linked polyvinyl pyrrolidone (crospovidone);
opacifying agents such as titanium dioxide;
buffering agents, including alkaline buffering agents, such as sodium hydroxide, sodium citrate, magnesium hydroxide, aluminum hydroxide, sodium carbonate, sodium bicarbonate, potassium phosphate, potassium carbonate, potassium bicarbonate, calcium phosphate, potassium hydroxide, calcium hydroxide, magnesium oxide, potassium dihydrogen phosphate, sodium dihydrogen phosphate, sodium phosphate, calcium carbonate, magnesium carbonate;
osmotic agents such as sodium chloride, calcium chloride, potassium chloride
diluents/tableting agents such as dicalcium phosphate and colloidal silicon dioxide;
antioxidants, including (1) water soluble antioxidants, such as ascorbic acid, cysteine hydrochloride, sodium bisulfate, sodium metabisulfite, and sodium sulphite, (2) oil-soluble antioxidants, such as ascorbyl palmitate, butylated hydroxyanisole (BHA), butylated hydroxytoluene (BHT), lecithin, propyl gallate, and alpha-tocopherol; and (3) metal chelating agents, such as citric acid, ethylenediamine tetraacetic acid (EDTA), tartaric acid, and phosphoric acid;
antibacterial and antifungal agents, such as paraben, chlorobutanol, phenol, sorbic acid;
mucosal adhesion enhancers such as starch graft copolymers (e.g., starch/acrylic acid copolymers) and other water-swellable polymers that adhere to wet surfaces of the oral mucosa such as carbomers, hydrolysed polyvinyl alcohol, polyethylene oxides, and polyacrylates;
as well as other non-toxic compatible substances employed in pharmaceutical formulations, such as liposomes and micelle forming agents;
including mixtures thereof.

Preferred rapidly infusing compositions are those which contain less than 1 wt. %, preferably less than 0.5 wt. %, preferably less than 0.1 wt. %, preferably less than 0.05 wt. %, preferably less than 0.001 wt. %, preferably 0 wt %, of other pharmaceutically acceptable carriers and/or excipients, such as those listed above, in particular alkaline buffering agents and/or surfactants.

Also preferred are rapidly infusing compositions which do not contain inert diluents, aqueous carriers, or non-aqueous carriers commonly used in the art for manufacture of liquid dosage forms for oral administration, such as emulsions, microemulsions, solutions, suspensions, syrups, and elixirs. Examples of inert diluents, aqueous or non-aqueous carriers, etc. which are preferably excluded herein may include, but are not limited to, water or other solvents, solubilizing agents, and emulsifiers, such as ethyl alcohol, isopropyl alcohol, ethyl carbonate, ethyl acetate, benzyl alcohol, benzyl benzoate, glycerol, polyethylene glycol, propylene glycol, 1,3-butylene glycol, oils (whether synthetic, semi-synthetic, or naturally occurring, such as long chain triglycerides, mixed glycerides, and free fatty acids, in particular, cottonseed oil, groundnut oil, corn oil, germ, olive oil, castor oil, sesame oil, borage oil, coconut oil, soybean oil, safflower oil, sunflower oil, palm oil, peanut oil, peppermint oil, poppy seed oil, canola oil, hydrogenated soybean oil, hydrogenated vegetable oils, glyceryl distearate, behenic acid, caprylyic/capric glycerides, lauric acid, linoleic acid, linolenic acid, myristic acid, palmitic acid, palmitoleic acid, palmitostearic acid, ricinoleic acid, stearic acid, soy fatty acids, oleic acid, glyceryl esters of fatty acids such as glyceryl behenate, glyceryl isostearate, glyceryl laurate, glyceryl palmitate, glyceryl palmitostearate, glyceryl ricinoleate, glyceryl oleate, glyceryl stearate), tetrahydrofuryl alcohol, fatty acid esters of sorbitan, organic esters such as ethyl oleate, and mixtures thereof, with specific mention being made to ethyl alcohol and sesame oil.

### Active Therapeutic Ingredient (ATI)

The amount of active therapeutic ingredient (ATI) which can be combined with the pharmaceutically acceptable carrier and/or excipient system to produce the rapidly infusing composition may vary depending upon the subject being treated, and other factors. The amount of ATI which can be combined with the pharmaceutically acceptable carrier and/or excipient system to produce a single dosage form will generally be that amount which produces a therapeutic effect. Generally, this amount will range from 0.1 to 90 wt. % of ATI, for example, at least 20 wt. %, preferably at least 22 wt. %, preferably at least 24 wt. %, preferably at least 26 wt. %, preferably at least 28 wt. %, preferably at least 30 wt. %, preferably at least 32 wt. %, preferably at least 34 wt. %, preferably at least 36 wt. %, preferably at least 38 wt. %, preferably at least 40 wt. %, preferably at least 42 wt. %, preferably at least 44 wt. %, preferably at least 46 wt. %, preferably at least 48 wt. %, preferably at least 50 wt. %, preferably at least 52 wt. %, preferably at least 54 wt. %, and up to 70 wt. %, preferably up to 68 wt. %, preferably up to 66 wt. %, preferably up to 64 wt. %, preferably up to 62 wt. %, preferably up to 60 wt. %, preferably up to 58 wt. %, preferably up to 56 wt. % of the ATI, based on a total weight of the rapidly infusing composition on a dry basis.

In terms of unit dose, the rapidly infusing composition is generally formulated with 2 to 100 mg of ATI per unit (e.g. tablet), for example at least 2 mg, preferably at least 4 mg, preferably at least 6 mg, preferably at least 8 mg, preferably at least 10 mg, preferably at least 12 mg, preferably at least 14 mg, preferably at least 16 mg, preferably at least 18 mg, preferably at least 20 mg, preferably at least 22 mg, preferably at least 24 mg, and up to 100 mg, preferably up to 75 mg, preferably up to 70 mg, preferably up to 65 mg, preferably up to 60 mg, preferably up to 55 mg, preferably up to 50 mg, preferably up to 45 mg, preferably up to 40 mg, preferably up to 35 mg, preferably up to 30 mg, preferably up to 25 mg of ATI per unit (e.g., tablet).

In preferred embodiments, the rapidly infusing composition is formulated with, as the active therapeutic ingredient, cannabidiol (CBD). In some preferred embodiments, CBD is the only active therapeutic ingredient in the rapidly infusing composition. In other embodiments, CBD may be combined with other active therapeutic ingredients. For example, CBD may be combined with a water-soluble ATI such as melatonin, as a sleep aid.

Preferred rapidly infusing compositions are those which are formulated with CBD, preferably a solid form of CBD. That is, the rapidly infusing composition is prepared through lyophilization from a drug product suspension in which the CBD is in the form of a solid. In particular, micronized particles of CBD are preferred. In some embodiments, the rapidly infusing composition is formulated with solid CBD in the form of micronized particles having a D50 particle size in the range of 1 μm to 50 μm, for example, those having a D50 particle size of at least 1 μm, preferably at least 10 μm, preferably at least 20 μm, preferably at least 30 μm, preferably at least 40 μm, and up to 50 μm, preferably up to 40 μm, preferably up to 30 μm, preferably up to 20 μm, preferably up to 10 μm.

Even more preferred are those rapidly infusing compositions which are formulated with a solid form of CBD having a purity of at least 95 wt. %, preferably at least 96 wt. %, preferably at least 97 wt. %, preferably at least 98 wt. %, preferably at least 99 wt. %. While CBD having a purity of 100 wt. % is likely not achievable, preferably rapidly infusing compositions are formulated with a solid form of CBD having a purity up to 99.1 wt. %, preferably up to 99.2 wt. %, preferably up to 99.3 wt. %, preferably up to 99.4 wt. %, preferably up to 99.5 wt. %, preferably up to 99.6 wt. %, preferably up to 99.7 wt. %, preferably up to 99.8 wt. %, preferably up to 99.9 wt. %. The percent purity of CBD refers to the percent of CBD by mass relative to a total weight of CBD containing material—the CBD containing material being the sum of CBD plus any additional impurities which may be present, such as those impurities originating from the biomass from which the CBD is obtained (e.g., Cannabis sativa L./“Industrial Hemp”) or encountered during manufacture. The purity may be determined by methods known to those of ordinary skill in the art, for example, one or more of liquid chromatography such as high performance liquid chromatography (HPLC), liquid chromatography-mass spectrometry (LCMS), and liquid chromatography with tandem mass spectrometry (LCMSMS); gas chromatography such as headspace gas chromatography with flame ionization detection (HS-GC-FID), gas chromatography mass spectrometry (GC/MS), and headspace gas chromatography-mass spectrometry (HSGCMS); inductively coupled plasma-mass spectrometry (ICP-MS); and polymers chain reaction (PCR).

Examples of potential impurities, such as those originating from the biomass from which the CBD is obtained (e.g., Cannabis sativa L./“Industrial Hemp”) or encountered during manufacture, include, but are not limited to,
cannabinoids (other than CBD) including, but not limited to, cannabidivarin (CBDV), cannabichromene (CBC), cannabidiolic acid (CBDa), cannabigerol (CBG), cannabigerolic acid (CBGa), cannabinol (CBN), tetrahydrocannabinolic acid (THCa), tetrahydrocannabivarin (TICV), tetrahydrocannabivarin acid (THCVa), and tetrahydrocannabinol (Δ9-THC) and related THC-cannabinoids such as Δ8-THC;
pesticides including, but not limited to, aldicarb, carbofuran, chlordane, chlorfenapyr, chlorpyrifos, coumaphos, daminozide, dichlorvos (DDVP), dimethoate, ethoprophos, etofenprox, fenoxycarb, fipronil, imazalil, methiocarb, methyl parathion, paclobutrazol, propoxur, spiroxamine, and thiacloprid;
residual solvents including, but not limited to, 1,4-dioxane, 2-butanol, 2-ethoxyethanol, 1,2-dichloroethane, acetone, acetonitrile, benzene, butane, cumene, cyclohexane, chloroform, ethanol, ethyl acetate, ethyl benzene, ethylene oxide, ethylene glycol, ethyl ether, heptane, isopropanol, methanol, methylene chloride, hexanes, isopropyl acetate, pentanes, propane, toluene, tetrahydrofuran, trichloroethene, and xylenes;
microbials including, but not limited to, Aspergillus flavus, Aspergillus fumigatus, Aspergillus niger, Aspergillus terreus, Salmonella, and Shiga toxin-producing E. coli;
mycotoxins including, but not limited to, aflatoxins (e.g., aflatoxin B1, aflatoxin B2, aflatoxin G1, and aflatoxin G2) and ochratoxin A;
heavy metals including, but not limited to, arsenic, cadmium, lead, and mercury;
terpenes including, but not limited to, (1) monoterpenes such as camphene, camphor, 3-carene, α-cedrene, cedrol, endo-fenchyl alcohol, eucalyptol, fenchone, geraniol, geranul acetate, hexahydrothymol, isoborneol, isopulegol, limonene, linalool, p-mentha-1,5-diene, β-myrcene, α- and β-pinene, pulegone, sabinene and hydrate, α- and γ-terpinene, terpineol, terpinolene, α-, β-, and γ-terpineol, nerol, borneol, and ocimene isomers I and II, and (2) sesquiterpenes such as α-bisabolol, β-caryophyllene, caryophyllene oxide, guaiol, α-humulene, cis- and trans-nerolidol, and valencene;
as well as mixtures thereof.

In some embodiments, the rapidly infusing composition is formulated with a form of CBD which contains less than 1 wt. %, preferably less than 0.5 wt. %, preferably less than 0.1 wt. %, preferably less than 0.05 wt. %, preferably less than 0.001 wt. %, preferably 0 wt. % of the above listed impurities, based on a total weight of the CBD material, with specific mention being made to THC. In some embodiments, the rapidly infusing composition is formulated with a form of CBD which contains no impurity, such as those listed above, in an amount above the limits of detection (LOD) and/or limits of quantification (LOQ) for the technique/instrumentation being used to make such a determination. For example, preferred rapidly infusing compositions are those formulated with a pure form of CBD which has a THC content of less than 0.1577 wt. %, preferably less than 0.1 wt. %, preferably less than 0.01 wt. %, preferably less than 0.001 wt. %, based on a total weight of the CBD material. In preferred embodiments, the rapidly infusing composition is formulated with a pure form of CBD which consists of, or consists essentially of, CBD.

The full effects of the present disclosure may not be realized when the rapidly infusing composition is formulated with an impure form of CBD or when the composition is formulated with CBD in oil/liquid form. Without being bound by theory, it is believed that during the manufacture of the rapidly infusing composition, when the CBD is in solid form with sufficiently high purity, lyophilization from a drug product suspension generates a structured and robust matrix of gelatin as the water is removed via sublimation, and an even distribution of the CBD throughout the gelatin matrix. Such a structured assembly of CBD suspended within a gelatin matrix is believed to afford the rapidly infusing composition with rapid disintegration properties and efficient transfer of CBD from the hydrophilic vehicle to the mucous membrane of the buccal cavity, or the ventral surface under the tongue, upon administration.

On the contrary, when the composition is formulated with an impure (oil) form of CBD during manufacture, lyophilization is instead performed from an o/w emulsion of CBD, which may produce an unstable, disordered matrix of gelatin more prone to collapse back into an oil or semi-solid state. The resulting composition tends to suffer from poor shelf-life, increased disintegration times, and inferior delivery/uptake of the CBD into systemic circulation reflected in longer onset times and overall less efficacy for the treatment of Tourette syndrome and tic disorders.

Accordingly, any CBD manufacturing method known by those of ordinary skill in the art which provides CBD in solid form, and of sufficient purity, may be utilized herein for preparation of the CBD ATI. For illustration purposes, one exemplary CBD manufacturing method is described below, although it should be understood that numerous modifications and variations are possible, and the CBD may be produced using methods or techniques otherwise than as specifically described.

CBD may be extracted/isolated from biomass, for example, a cured flower of Cannabis sativa L. The biomass may contain, for example, at least 1 mg/g, preferably at least 2 mg/g, preferably at least 3 mg/g, and up to 10 mg/g, preferably up to 8 mg/g, preferably up to 6 mg/g, preferably up to 4 mg/g of CBD; at least 50 mg/g, preferably at least 60 mg/g, preferably at least 70 mg/g, preferably at least 80 mg/g, preferably at least 90 mg/g, and up to 150 mg/g, preferably up to 140 mg/g, preferably up to 130 mg/g, preferably up to 120 mg/g, preferably up to 110 mg/g, preferably up to 100 mg/g of cannabidiolic acid (CBDa); and no detectable amount of THC. Extraction of the biomass with an alcoholic solvent (e.g., ethanol) and cooling may form a tincture. The tincture may be filtered to remove sediment and particulates, and concentrated, for example, using a rotary evaporator.

An aluminum phyllosilicate clay (e.g., bentonite) may then be mixed with the concentrated product at a weight ratio of at least 2:1, preferably at least 3:1, preferably at least 4:1, and up to 6:1, preferably up to 5:1, and the resulting mix filtered to remove fats, waxes, and lipids. The product may then be frozen/winterized, after which the frozen product may be again filtered and taken through another solvent removal/recovery cycle to form a winterized crude.

Decarboxylation of the winterized crude by heating, for example in an induction oven centrifugal reactor, may be performed to remove the carboxylic acid functionality from the cannabinoids. Distillation of the decarboxylated material may then provide a distillate.

The distillate may then be precipitated in a high-pressure reactor using an alkane solvent (e.g., pentane), and a cryochamber may be used to subject the precipitate to cryo temperatures (e.g., −20° F. to −40° F.) to promote the growth of crystalline CBD. The CBD crystals may be washed with an alkane solvent (e.g., pentane), filtered, and ground to a finer particle size, prior to being purged in a vacuum oven for removal of solvents and impurities. The obtained solid CBD may then be analyzed for purity, as appropriate.

In preferred embodiments, the rapidly infusing composition comprises, consists essentially of, or consists of gelatin, mannitol, sweetener, flavorant, colorant, and as the ATI. CBD.

Also contemplated for use as an active therapeutic ingredient are derivatives/analogs of CBD that retain the desired activity for the treatment of Tourette syndrome and tic disorders. Derivatives/analogs that retain substantially the same activity as CBD, or more preferably exhibit improved activity, may be produced according to standard principles of medicinal chemistry, which are well known in the art. Such derivatives/analogs may exhibit a lesser degree of activity than CBD, so long as they retain sufficient activity to be therapeutically effective. Derivatives/analogs may exhibit improvements in other properties that are desirable in active therapeutic agents such as, for example, improved solubility, reduced toxicity, enhanced uptake, increased bioavailability, etc. Contemplated CBD derivatives/analogs include, but are not limited to, cannabidiolic acid compounds and variants thereof, such as cannabidiolic acid and esters of cannabidiolic acid, in particular alkyl esters of cannabidiolic acid (e.g., cannabidiolic acid methyl ester); 5′ side chain modified CBD compounds such as cannabidivarin (CBDV), cannabidiol-dimethylheptyl (CBD-DMH), and 1,2-cannabidiol-dimethylheptyl (1,2-CBD-DMH); 7-methyl modified CBD compounds such as 7-carboxy cannabidiol (7-COOH-CBD) and 7-hydroxy cannabidiol (7-OH-CBD); hydrogenated CBD compounds such as 8,9-dihydrocannabidiol (HrCBD) and tetrahydrocannabidiol (H₄—CBD); halogenated CBD compounds such as 3′-chloro-CBD, 3′,5′-dichloro-CBD, 3′-bromo-CBD, 3′,5′-dibromo-CBD, 3′-iodo-CBD, and 3,5′-diiodo-CBD; hydroxyl group modified CBD compounds such as desoxy-CBD and dimethylether CBD; cannabielsoin (CBE); machaeridiols A, B, and C; as well as any pharmaceutically acceptable salts, solvates, and/or stereoisomers of such compounds. When a CBD derivative/analog is used as the ATI in the disclosed rapidly infusing composition, particular preference is given to 7-hydroxy cannabidiol (7-OH-CBD). It is contemplated that CBD or derivatives/analogs of CBD may be useful in combination. It is also contemplated that CBD or derivatives/analogs of CBD may be useful in combination with current Standards of Care for the treatment of Tourette syndrome and tic disorders as well as any treatments that evolve over the foreseeable future. Specific dosages and dosing regimens would be based on physicians' evolving knowledge and the general skill in the art.

### Process for Manufacturing the Rapidly Infusing Composition

Manufacturing of the rapidly infusing compositions are preferably pharmaceutical-GMP compliant and may be accomplished generally by bringing into association the ATI (e.g., CBD) with the gelatin and sugar alcohol (e.g., mannitol), and, optionally, one or more accessory pharmaceutically acceptable carrier and/or excipient ingredients, in water to form a drug product suspension which is then lyophilized.

One exemplary method for manufacturing the rapidly infusing composition is presented below, although it should be understood that numerous modifications and variations are possible, and the rapidly infusing composition may be produced using methods or techniques otherwise than as specifically described.

Purified water, gelatin, and sugar alcohol (e.g., mannitol) may be charged to a mixer, for example a pot equipped with an overhead stirrer, and heated (e.g., 40 to 80° C.) with agitation until complete solvation. Any desired sweetener (e.g., a mixture of sucralose and acesulfame-K) may then be added and allowed to dissolve.

Upon cooling, for example to 20 to 35° C., the solution may next be transferred to a homogenizer, and the ATI (e.g., CBD) may be subsequently charged and dispersed using the homogenizer, with preferable micronization of the ATI, to form a drug product suspension. Any desired flavorant and colorant may be added at this point with continued mixing. The drug product suspension may be transferred to a second mixer whilst maintaining a cooled temperature (e.g., 20 to 35° C.).

In a blistering machine equipped with a dosing system, blister pockets may next be filled with the drug product suspension until achieving a target dose weight, followed by freezing in a suitable cryochamber. The blister trays may be transferred from the cryochamber to a suitable refrigerated storage cabinet (e.g., at a temperature below 0° C.) to keep the product frozen prior to lyophilization. Then, the frozen blisters may be loaded into a lyophilizer and subject to lyophilization to sublimate the water and form the rapidly infusing compositions. Finally, when the lyophilization cycle is deemed complete, final sealing (e.g., heat sealing of blister lidding) may be performed to provide the rapidly infusing compositions in single dose units in individual blister units.

### Therapeutic Applications and Methods

The present disclosure provides a method of treating Tourette syndrome and/or tic disorders by administering to a subject in need thereof the disclosed rapidly infusing composition, in one or more of its embodiments. A person having Tourette syndrome and/or tic disorders presents with tics, which are abnormal involuntary movements. Tics can take many forms, but typically consist of simple, repetitive or sequential movements, gestures, and utterances that mimic fragments of normal behavior.

In one embodiment, the subject presents with a symptom of an abnormal involuntary movement including but not limited to an eye movement (e.g. excessive blinking, squinting, moving eyes in a certain direction, rolling eyes), a facial movement (e.g. a grimace, smacking lips, raising or lowering sides of mouth, twitching ears, jaw opening, broadening nostrils, sticking out tongue), a shoulder movement (e.g. shrugging), a head movement (e.g. nodding, shaking, turning), a respiratory movement (e.g. sniffing, exhaling, sighing, coughing, throat clearing), and a vocalization (e.g. grunting, squeaking, moaning, screaming, speaking).

In one embodiment, the subject presents with a complex symptom, which may be a combination of two or more of the above symptoms occurring sequentially (with little or no pause in between) or simultaneously. In one embodiment, the complex symptom is a speech-related symptom including but not limited to coprolalia (uttering swear words), echolalia (repeating the words or phrases of others), and palilalia (repeating one's own utterances, particularly the last syllable, word, or phrase in a sentence). In one embodiment, the complex symptom is a motor-related symptom involving several muscle groups, including but not limited to hopping, jumping, bending, twisting, brushing hair, throwing objects, touching objects, copropraxia (making lewd or obscene gestures), and echopraxia (imitating others' gestures).

In one embodiment, the subject has Tourette syndrome. In another embodiment, the subject does not have Tourette syndrome but has a tic disorder including but not limited to pediatric autoimmune disorder associated with streptococcal infection (PANDAS), a transient tic disorder, and a chronic tic disorder.

Transient tic disorders are generally characterized by multiple motor and/or phonic tics that occur for at least four weeks but less than 12 months. Chronic tic disorders are generally characterized by either single or multiple motor or phonic tics, but not both, which are present for more than a year; Tourette syndrome may be diagnosed when both motor and phonic tics are present (although not necessarily concurrently) for more than one year. In some instances, a patient may present with a tic disorder but may not be diagnosed with Tourette syndrome due to differences in diagnostic criteria relating to the number, intensity, duration, severity, frequency, or other nature of the tics. The patient may have a “tic disorder not otherwise specified” in this case if tics are present but do not meet the criteria for any specific tic disorder or neurodegenerative disorder.

In one embodiment, the subject may a movement disorder which causes tic-like symptoms in addition to or instead of Tourette syndrome. The movement disorder may be considered a tic disorder. Movement disorders include but are not limited to akathisia, akinesia, ataxia, athetosis, ballismus, bradykinesia, cerebral palsy, chorea, corticobasal degeneration, dyskinesias (e.g., paroxysmal), dystonia (general, segmental, focal) including blepharospasm, writer's cramp (limb dystonia), laryngeal dystonia (spasmodic dysphonia), and oromandibular dystonia, tremor, essential tremor, geniospasm, hereditary spastic paraplegia, Huntington's Disease, multiple system atrophy (Shy Drager Syndrome), myoclonus, Parkinson's Disease, Parkinson's disease levodopa-induced dyskinesia, parkinsonism, progressive supranuclear palsy, restless legs syndrome, Rett Syndrome, spasmodic torticollis (cervical dystonia), spasticity due to stroke, multiple sclerosis, spinal cord or brain injury, stereotypic movement disorder, stereotypy, Sydenham's Chorea, synkinesis, tardive dyskinesia, neuroacanthocytosis, pantothenate kinase-associated neurodegeneration, Duchenne muscular dystrophy, and Wilson's Disease. Other possibilities include chromosomal disorders such as Down syndrome, Klinefelter syndrome, XYY syndrome, fragile X syndrome, and Lesch-Nyhan syndrome. Acquired causes of tics include drug-induced tics, head trauma, encephalitis, stroke, and carbon monoxide poisoning.

In one embodiment, the subject may have another condition, including but not limited to stuttering, attention deficit hyperactivity disorder, obsessive-compulsive disorder, or autism spectrum disorder.

With respect to administration, the rapidly infusing composition is preferably administered to the subject via one or more of the oral mucosae, preferably via the buccal mucosa (buccally) or the sublingual mucosa (sublingually). Advantages of oral mucosal delivery include the ease of administration, the ability to bypass first pass metabolic processes thereby enabling higher bioavailability than through enteral delivery via the gastrointestinal tract (which in turn allows the dosage amount of ATI to be reduced whilst maintaining the same pharmacological effect), less variability between patients, sustained drug delivery, and extensive drug absorption and rapid onset of therapeutic action due to either a large surface area in the case of sublingual administration or high-levels of vascularization in the case of buccal administration. Administration may be carried out by simply placing the rapidly infusing composition directly in the buccal cavity (between the cheek and gum) or over the sublingual mucous gland (under the ventral surface of the tongue). While the sublingual mucosa has a large surface area and extremely good permeability, the blood supply (blood flow) is lesser than that of the buccal cavity. Furthermore, sublingual administration tends to stimulate the flow of saliva more than buccal administration, and the increased saliva production may make it more difficult for patients to avoid swallowing. Any amount of ATI that is swallowed would be subject to first pass metabolism and thus overall lower bioavailability. Swallowing further results in greater variability in the effective amount of dosing, as a result of, including but not limited to, the variability in the amount swallowed and the greater patient variability of bioavailability through first-pass metabolism for the amount swallowed. Therefore, in preferred embodiments, the rapidly infusing composition is administered buccally (through the buccal mucosa). The rapid disintegration of the rapidly infusing composition, approximately in 1-5 seconds in preferred embodiments, and buccal administration together combine to provide optimal dosing control by limiting the time for potential swallowing and ensuring that the vast majority of the ATI is absorbed through the buccal mucosa. Administration may be performed by the subject (self-administered) or by someone other than the subject, for example, a healthcare provider, care-taker, family member, etc.

In one embodiment, the administration may be carried out in the absence of an environmental modification comprising a trigger. A trigger may cause a premonitory urge in the subject to exhibit a tic, or may more directly cause or lead to a tic. Such trigger may be a noisy environment or certain noises, crowds, electronic screen media, lighting (such as fluorescent lights, flickering or flashing lights, or blue-shifted light), dust, pollen, other airborne allergens, and strong odors. Other triggers may be dietary or medicinal, including but not limited to gluten, dairy, refined carbohydrates including sugars, caffeine, and ADHD medications.

The actual amount of ATI administered to the subject may be varied so as to achieve the desired therapeutic response for a particular subject, composition, and mode of administration, without being toxic to the subject. The selected amount of ATI administered to the subject will depend upon a variety of factors including the condition being treated, the activity of the ATI employed, the route of administration, the time of administration, the rate of excretion or metabolism of the particular compound being employed, the rate and extent of absorption, the duration of the treatment, other drugs, compounds, and/or materials used in combination with the rapidly infusing composition, the age, sex, weight, condition, general health, and prior medical history of the subject being treated, and like factors well known in the medical arts.

A physician having ordinary skill in the art can readily determine and prescribe the effective amount of the ATI required. For example, the physician could start doses of the ATI at levels lower than that required in order to achieve the desired therapeutic effect and gradually increase the dosage until the desired effect is achieved. In general, a suitable dose of the ATI will be that amount which is the lowest dose effective to produce a therapeutic effect, which will generally depend upon the factors described above. Typically, when the ATI is CBD, the therapeutically effective amount of CBD will be under 7 mg/kg/day, for example in a range of from at least 0.1 mg/kg/day, preferably at least 0.15 mg/kg/day, preferably at least 0.2 mg/kg/day, preferably at least 0.25 mg/kg/day, preferably at least 0.3 mg/kg/day, preferably at least 0.35 mg/kg/day, preferably at least 0.4 mg/kg/day, preferably at least 0.45 mg/kg/day, preferably at least 0.5 mg/kg/day, preferably at least 0.55 mg/kg/day, preferably at least 0.6 mg/kg/day, and up to 6.9 mg/kg/day, preferably up to 6.8 mg/kg/day, preferably up to 6.6 mg/kg/day, preferably up to 6.4 mg/kg/day, preferably up to 6.2 mg/kg/day, preferably up to 6 mg/kg/day, preferably up to 5.5 mg/kg/day, preferably up to 5 mg/kg/day, preferably up to 4.5 mg/kg/day, preferably up to 4 mg/kg/day, preferably up to 3.5 mg/kg/day, preferably up to 3 mg/kg/day, preferably up to 2.5 mg/kg/day, preferably up to 2 mg/kg/day, preferably up to 1.5 mg/kg/day, preferably up to 1.25 mg/kg/day, preferably up to 1 mg/kg/day, preferably up to 0.9 mg/kg/day, preferably up to 0.8 mg/kg/day, preferably up to 0.7 mg/kg/day, preferably up to 0.65 mg/kg/day.

In one embodiment, the therapeutically effective amount of CBD or a thereof will be 7 mg/kg/day or less, preferably up to 6.5 mg/kg/day, preferably up to 6 mg/kg/day, preferably up to 5.5 mg/kg/day, preferably up to 5 mg/kg/day. 00871 In a preferred embodiment, CBD is the only active therapeutic ingredient in the rapidly infusing composition, and the rapidly infusing composition is substantially free of other CBD derivatives and CBD analogs. Here, that the composition has a content of CBD derivatives and analogs of less than preferably less than 0.1 wt. %, preferably less than 0.01 wt. %, preferably less than 0.001 wt. %, or about 0 wt. % based on a total weight of the CBD. In a further embodiment, the CBD is the only active therapeutic ingredient in the rapidly infusing composition and the subject is not administered a cannabinoid other than CBD (for instance, by co-administration).

The methods herein may involve administering one, or more than one, unit of the rapidly infusing composition per dose (dosing event). For example, in circumstances where each unit of the rapidly infusing composition contains 25 mg of ATI (e.g., CBD), and it has been determined that a subject weighing 75 kg requires a therapeutically elective amount of 2 mg/kg/day of ATI, then the subject may be given two (2) units (e.g., tablets) three times a day (t.i.d.) to achieve the desired therapeutically effective amount of 2 mg/kg/day. In another example, in circumstances where each unit of the rapidly infusing composition contains 10 mg of ATI (e.g., CBD), and it has been determined that a subject weighing 20 kg requires a therapeutically effective amount of 2 mg/kg/day of ATI, then the subject may be given two (2) units (e.g., tablets) twice a day (b.i.d.) to achieve the desired therapeutically effective amount of 2 mg/kg/day. Accordingly, depending on the unit dose of ATI in each unit of the rapidly infusing composition, the therapeutically effective amount of ATI prescribed, etc., 1, 2, 3, 4, 5, or more units (e.g., tablets) may be administered to the subject per dose. Accordingly, the phrases “administering to the subject in need thereof a rapidly infusing composition”, “the rapidly infusing composition is administered”, etc., are intended herein to include administration of a single unit (e.g., tablet), or multiple units (e.g., tablets), to the subject in order to provide the therapeutically effective amount of AT, e.g., CBD. While it may be possible to administer partial (e.g., half) tablets to the subject, for practical reasons, it is preferred that one or more whole tablets are administered to the subject.

In preferred embodiments, the subject may be prescribed a dosage regimen that involves a single dosing event per day (QD), or multiple, separate dosing events at appropriate time intervals throughout the day. The subject may be administered a therapeutically effective amount of ATI 1 time, 2 times, 3 times, 4 times, 5 times, 6 times, 7 times, 8 times, 9 times, or 10 times at appropriate intervals, throughout the day. Preferred dosing regimens involve administration at the same time each day, for example, following meal times every morning and/or evening. Particularly preferred dosing schedules involve administration of the rapidly infusing composition once (QD), two times (b.i.d.), or three times (t.i.d.) per day. The rapidly infusing composition may also be administered on an hourly dosing schedule (q), for example, administration may take place every 8 to 12 hours, as appropriate. Regardless of dosing schedule, when the ATI is CBD, the maximum daily dosage of CBD is preferably less than 7 mg/kg/day. Treatment may involve administration until desired effects are achieved, for example for weeks, months, or even years, or throughout the subjects life-span.

Preferred dosing regimens are those involving a consistent dosing amount and schedule. One non-limiting example of a dosing regimen may involve the subject taking one unit of the rapidly infusing composition (e.g., 25 mg CBD)—therapeutically effective amount of 25 mg CBD per dose-once per day (QD). Another non-limiting example of a dosing regimen may involve the subject taking one unit of the rapidly infusing composition (e.g., 25 mg CBD)—therapeutically effective amount of 25 mg CBD per dose-two times per day (b.i.d.). Another non-limiting example of a dosing regimen may involve the subject taking two units of the rapidly infusing composition (e.g., 10 mg CBD each)—therapeutically effective amount of 20 mg CBD per dose-two times per day (b.i.d.).

Upon being administered buccally (between the cheek and gum) or sublingually (under the ventral surface of the tongue), the rapidly infusing composition preferably disintegrates in 5 seconds or less, preferably 4 seconds or less, preferably 3 seconds or less, preferably 2 seconds or less, preferably about 1 second. Further, this route of administration may provide a single dose bioavailability of at least 20%, preferably at least 30%, preferably at least 40%, preferably at least 50%, preferably at least 60%, preferably at least 70%, preferably at least 80%, preferably at least 90%, and up to 95%, preferably up to 90%, preferably up to 80%, preferably up to 70%, preferably up to 60%, preferably up to 55%, preferably up to 40%, preferably up to 30%.

Besides efficacy of treatment and general relief from Tourette syndrome and tic disorders, pharmacokinetic outcomes may provide another useful measure of in vivo performance. In this regard, the rapidly infusing composition formulated with CBD and administered according to the methods described herein may provide a time to maximum plasma concentration (Tmax) of less than 5 hours, preferably less than 4.5 hours, preferably less than 4.25 hours, preferably less than 4 hours, preferably less than 3.75 hours, preferably less than 3.5 hours, preferably less than 3.25 hours, preferably less than 3 hours, preferably less than 2.75 hours, preferably less than 2.5 hours, preferably less than 2.25 hours, preferably less than 2 hours, preferably less than 1.75 hours, preferably less than LS hours, preferably less than 1.25 hours, preferably less than 1 hour, preferably less than 45 minutes, preferably less than 30 minutes, preferably less than 15 minutes; an area under the plasma concentration versus time curve (AUC) of at least 1 h×ng/mL, preferably at least 3 h×ng/mL, preferably at least 5 h×ng/mL, preferably at least 10 h×ng/mL, preferably at least 15 h×ng/mL, preferably at least 20 h×ng/mL, preferably at least 25 h×ng/mL, preferably at least 30 h×ng/mL, and up to 80 h×ng/mL, preferably up to 70 h×ng/mL, preferably up to 60 h×ng/mL, preferably up to 50 h×ng/mL, preferably up to 40 h×ng/mL, from a single (1) unit of rapidly infusing composition formulated with 25 mg CBD; and a mean plasma half-life (t_{1/2}) of CBD of at least 1 hour, preferably at least 2 hours, preferably at least 3 hours, preferably at least 4 hours, preferably at least 5 hours, preferably at least 6 hours, and up to 12 hours, preferably up to 11 hours, preferably up to 0.10 hours, preferably up to 9 hours, preferably up to 8 hours, preferably up to 7 hours, for a single dose, but may provide a significantly higher mean plasma half-life (t_{1/2}) after prolonged buccal or sublingual administration (e.g., t_{1/2} of 2 to 5 days).

In one embodiment, an effective treatment for Tourette syndrome and tic disorders, or an improvement in Tourette syndrome and tic disorders, could manifest as a decrease in the number, duration, frequency, complexity, interference, and/or intensity of any of the aforementioned tics and abnormal involuntary movements. Here, interference refers to the degree that the tic or tics directly disrupt the intended actions and behaviors of the patient. In other embodiments, the improvement may manifest more subjectively in terms of an increased well-being or physical ability of the subject. In one embodiment, a tic number, duration, frequency, complexity, interference, and/or intensity in the subject is reduced by preferably at least 5%, preferably at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, or preferably 100/o, and/or preferably up to 100%, preferably up to 95%, preferably up to 90%, preferably up to 85%, preferably up to 80%, preferably up to 75%, preferably up to 70%, preferably up to 65%, preferably up to 60%, preferably up to 55%, preferably up to 50%, preferably up to 45%, preferably up to 40%, preferably up to 35%, preferably up to 30%, preferably up to 25%, preferably up to 20%, preferably up to 15%, preferably up to 10%, preferably up to 5% relative to the number, duration, frequency, complexity, interference, and/or intensity observed prior to administration of the rapidly infusing composition. Here, a reduction in tic number, duration, frequency, and/or intensity of 100% also means that the occurrence of tics in the subject is reduced completely. The reduction in tic number, duration, frequency, complexity, interference, and/or intensity may come by administering the rapidly infusing composition for a period of time of preferably at least 15 minutes, preferably at least 30 minutes, preferably at least 45 minutes, preferably at least 1 hour, preferably at least 1.5 hours, preferably at least 2 hours, preferably at least 3 hours, preferably at least 4 hours, preferably at least 6 hours, preferably at least 8 hours, preferably at least 12 hours, preferably at least 18 hours, preferably at least 1 day, preferably at least 2 days, preferably at least 3 days, preferably at least 1 week, preferably at least 2 weeks, preferably at least 4 weeks, preferably at least 1 month, preferably at least 2 months, preferably at least 3 months, preferably at least 4 months, preferably at least 6 months, preferably at least 8 months, preferably at least 10 months, preferably at least 12 months, preferably at least 18 months and/or preferably up to 18 months, preferably up to 12 months, preferably up to 10 months, preferably up to 8 months, preferably up to 6 months, preferably up to 4 months, preferably up to 3 months, preferably up to 2 months, preferably up to 1 months, preferably up to 4 weeks, preferably up to 2 weeks, preferably up to 1 week, preferably up to 3 days, preferably up to 2 days, preferably up to 1 day, preferably up to 18 hours, preferably up to 12 hours, preferably up to 8 hours, preferably up to 6 hours, preferably up to 4 hours, preferably up to 3 hours, preferably up to 2 hours, preferably up to, hour.

In one embodiment, the improvement may be measured or reflected in a patient evaluation scale. The evaluation scale may be administered by the subject or by a medical professional observing the subject undergoing treatment. Evaluation scales include but are not limited to the Yale Global Tic Severity Scale (YGTSS), the Tourette Syndrome Patient Global Impression of Severity (TS-PGIS), Tourette Syndrome Clinical Global Impression (TS-CGI), the Tic-Free Interval scale, Tourette Syndrome Patient Global Impression of Change (TS-PGIC), and the Gilles de la Tourette Syndrome-Quality of Life Scale (GTS-QOL). Other scales that may measure improvements in patient outcomes not directly related to tic severity and frequency include but are not limited to the SF-36 Physical Functioning Score and the Premonitory Urge for Tics Scale (PUTS).

The Yale Global Tic Severity Scale (YGTSS) is a comprehensive evaluation of various aspects and severity of motor and phonic tics. In one aspect, each of five categories-number, frequency, intensity, complexity, and interference—is scored from 0 to 5 for both motor and phonic tics, producing a tic severity score of 0 to 25 for each of Vocal Tic Severity Score (VTSS) and Motor Tic Severity Score (MTSS). Added together, these comprise the total tic severity (TTS) score. Separately, impairment of the patient's life is scored on a scale of 0 to 50, wherein 0 indicates no impairment, 10 is minimal, 20 is mild, 30 is moderate, 40 is marked, and 50 is severe, yielding an impairment score. When the Impairment score is added to the TTS score, this comprises the complete Global Severity Score (GSS) of the YGTSS.

In one embodiment, a tic severity of the subject as measured by the Yale Global Tic Severity Scale is reduced by preferably at least 5%, preferably at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, or preferably 100%, and/or preferably up to 100%, preferably up to 95%, preferably up to 90%, preferably up to 85%, preferably up to 80%, preferably up to 75%, preferably up to 70%, preferably up to 65%, preferably up to 60%, preferably up to 55%, preferably up to 50%, preferably up to 45%, preferably up to 40%, preferably up to 35%, preferably up to 30%, preferably up to 25%, preferably up to 20%, preferably up to 15%, preferably up to 10%, preferably up to 5% relative to the tic severity prior to administration of the rapidly infusing composition.

The Tourette Syndrome Patient Global Impression of Severity (TS-PGIS) is a five-point scale in which 1 indicates no tics, 2 indicates mild tics (not distressing, noticeable, or interfering with daily life), 3 indicates moderate (can be distressing, noticeable, and sometimes interfering with daily life), 4 indicates marked (very distressing, noticeable, and interfering with daily life), and 5 indicates severe (severely distressing, always noticeable, and preventing of most daily activities).

The Tourette Syndrome Clinical Global impression (TS-CGI) is a seven-point scale scored by the clinician, in which 1 indicates normal or no tics, 2 indicates tics may or may not be present, 3 indicates mild, observable motor and/or phonic tics that may or may not be noticed, would not call attention to the individual, and are associated with no distress or impairment, 4 indicates moderate, observable motor and/or phonic tics that would always be noticed, would call attention to the individual, and may be associated with some distress or impairment, 5 indicates marked, exaggerated motor and/or phonic tics that are disruptive, would always call attention to the individual, and are always associated with significant distress or impairment, 6 indicates severe, extremely exaggerated motor and/or phonic tics that are disruptive, would always call attention to the individual, and are associated with injury or inability to carry out daily functions, and 7 indicates extreme, incapacitating tics.

In one embodiment, the TS-PGIC and/or the TS-CGI is reduced by preferably at least 1 point, preferably at least 2 points, preferably at least 3 points, preferably at least 4 points, preferably at least 5 points, preferably at least 6 points, and/or preferably up to 6 points, preferably up to 5 points, preferably up to 4 points, preferably up to 3 points, preferably up to 2 points, preferably up to 1 point relative to the TS-PGIC and/or the TS-CGI prior to administration of the rapidly infusing composition.

The Tic-Free Interval is a five-point scale in which 1 indicates an interval of at least one day since the last tic, 2 indicates an interval of between 6 hours to less than one day since the last tic, 3 indicates an interval of between one hour and less than 6 hours since the last tic, 4 indicates an interval of between five minutes to less than one hour since the last tic, and 5 indicates less than five minutes since the last tic. In one embodiment, the Tic-Free Interval of the subject is reduced by preferably at least 1 point, preferably at least 2 points, preferably at least 3 points, preferably at least 4 points relative to the Tic-Free Interval prior to administration of the rapidly infusing composition.

The Tourette Syndrome Patient Global Impression of Change (TS-PGIC) is a seven-point scale in which −3 indicates very much worse, −2 indicates much worse, −1 indicated minimally worse, 0 indicated no change, 1 indicates minimally improved, 2 indicates much improved, and 3 indicates very much improved. In one embodiment, the subject indicates preferably a 1, preferably a 2, preferably a 3 on the TS-PGIC scale following administration of the rapidly infusing composition, where the change is compared with the subject's condition prior to administration.

The Gilles-de-la-Tourette-Syndrome Quality-of-Life scale (GTS-QOL) consists of two parts. The first part is typically a twenty-seven question assessment of various aspects of how tics affect the subject's life, each to be rated on a five-choice scale of no problem, slight problem, moderate problem, marked problem, or severe problem. Subscales combining some of these aspects can be focused upon, such as, e.g., the physical/activities of daily living subscale. The second part is a simple rating of the subject's life satisfaction, where 100 is extremely satisfied and 0 is extremely dissatisfied. In one embodiment, the scale of the first part and/or the second part is improved by preferably at least 10%, preferably at least 20%, preferably at least 30%, preferably at least 40%, preferably at least 50%, preferably at least 60%, preferably at least 70%, preferably at least 80%, preferably at least 90% relative to the scale prior to administration of the rapidly infusing composition.

The Premonitory Urge for Tics Scale (PUTS) is used by a subject to objectify the premonitory urge, which is the sensory phenomenon preceding, encouraging, or pushing towards a tic. Sensory phenomena in tic disorders include bodily sensations, mental urges, and a sense of inner tension, feelings of incompleteness, and a need for things to be “just right.” Bodily sensations include focal or generalized body sensations (usually tactile, muscular-skeletal/visceral, or both); mental sensations include urge only, energy release (mental energy that builds up and needs to be discharged), incompleteness, and just-right perceptions. In contrast to the stereotyped movements of other movement disorders such as choreas, dystonias, myoclonus, and dyskinesias, the tics of Tourette's are temporarily suppressible and preceded by this premonitory urge. The PUTS is a self-evaluation where the total score scales from 9 (lowest or no premonitory urge) to 36 (highest premonitory urge or urges). See Woods, D W et al. J Dev Behav Pediat. 2005; 26 (6): 397-403—incorporated herein by reference in its entirety.

In one embodiment, a premonitory urge for tics of the subject as measured by a the Premonitory Urge for Tics Scale (PUTS) is reduced by preferably at least 5%, preferably at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, or preferably 100%, and/or preferably up to 100%, preferably up to 95%, preferably up to 90%, preferably up to 85%, preferably up to 80%, preferably up to 75%, preferably up to 70%, preferably up to 65%, preferably up to 60%, preferably up to 55%, preferably up to 50%, preferably up to 45%, preferably up to 40%, preferably up to 35%, preferably up to 30%, preferably up to 25%, preferably up to 20%, preferably up to 15%, preferably up to 10%, preferably up to 5% relative to the premonitory urge for tics prior to administration of the rapidly infusing composition.

Using the platform, the rapidly infusing composition may be used as a stand-alone therapeutic agent for the treatment of Tourette syndrome and tic disorders or may be used in combination therapy—wherein the rapidly infusing composition is used in combination with one or more other forms of therapy such as one or more second therapeutic agents. The combination therapy may be applied to treat Tourette syndrome and tic disorders, or a combination of Tourette syndrome and a different condition or a tic disorder and a different condition.

Combination therapy may involve administering the rapidly infusing composition formulated with CBD and a second therapeutic agent, other than CBD, for the treatment of Tourette syndrome and tic disorders. In particular, when co-administered with the second therapeutic agent, the rapidly infusing compositions formulated with CBD may advantageously function to decrease usage of the second therapeutic agent, for example by reducing the number of therapeutic agents dosed, reducing the overall dosage of the second therapeutic agent, or shortening the dosage period, without sacrificing the drug's effect.

In one embodiment, the rapidly infusing composition is administered in combination with at least one drug selected from the group consisting of a dopamine precursor, a DOPA decarboxylase inhibitor, a catechol-O-methyl transferase (COMT) inhibitors, a dopamine receptor agonist, a neuroprotective agent, an NMDA antagonist, an anti-psychotic, a benzodiazepine, and a CYP2D6 inhibitor.

In one embodiment, the rapidly infusing composition is administered in combination with a dopamine precursor including but not limited to levodopa.

In one embodiment, the rapidly infusing composition is administered in combination with a DOPA decarboxylase inhibitor including but not limited to carbidopa.

In one embodiment, the rapidly infusing composition is administered in combination with a catechol-O-methyl transferase (COMT) inhibitors including but not limited to entacapone, opicapone, nebicapone, nitecapone, and tolcapone.

In one embodiment, the rapidly infusing composition is administered in combination with a dopamine receptor agonist including but not limited to apomorphine, bromocriptine, ropinirole, and pramipexole.

In one embodiment, the rapidly infusing composition is administered in combination with a neuroprotective agent including but not limited to selegeline and riluzole.

In one embodiment, the rapidly infusing composition is administered in combination with an NMDA antagonist including but not limited to amantadine.

In one embodiment, the rapidly infusing composition is administered in combination with an anti-psychotic drug including but not limited to chlorpromazine, levomepromazine, promazine, acepromazine, triflupromazine, cyamemazine, chlorproethazine, dixyrazine, fluphenazine, perphenazine, prochlorperazine, thiopropazate, trifluoperazine, acetophenazine, thioproperazine, butaperazine, perazine, periciazine, thioridazine, mesoridazine, pipotiazine, haloperidol, fluphenazine, trifluperidol, melperone, moperone, pipamperone, bromperidol, benperidol, droperidol, fluanisone, oxypertine, molindone, sertindole, ziprasidone, flupentixol, clopenthixol, chlorprothixene, thiothixene, zuclopenthixol, fluspirilene, pimozide, penfluridol, loxapine, clozapine, olanzapine, quetiapine, tetrabenazine, sulpiride, sultopride, tiapride, remoxipride, amisulpride, veralipride, levosulpiride, lithium, prothipendyl, risperidone, clotiapine, mosapramine, zotepine, pripiprazole, and paliperidone.

In one embodiment, the rapidly infusing composition is administered in combination with a benzodiazepine including but not limited to alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

In one embodiment, the rapidly infusing composition is administered in combination with a CYP2D6 inhibitor including but not limited to amiodarone, celecoxib, chloroquine, chlorpromazine, cimetidine, citalopram, clomipramine, codeine, deiavirdine, desipramine, dextroprpoxyphene, diltiazem, doxorubicin, entacapone, fluoxetine, fluphenazine, fluvaxamine, haloperidol, labetalol, lobeline, lomustine, methadone, mibefradil, moclobemide, nortuloxeline, paroxetine, perphenazine, propafenone, quinacrine, quinidine, ranitidine, risperidone, ritonavir, serindole, sertraline, thioridazine, valproic acid, venlafaxine, vinblastine, vincristine, vinorelbine, and yohimbine. In one embodiment, the CYP2D6 inhibitor is a strong CYP2D6 inhibitors including but not limited to fluoxetine, paroxetine, bupropion, quinidine, cinacalcet, and ritonavir.

In another embodiment, the rapidly infusing composition may be administered with some other drug or compound, including but not limited to anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs), such as naproxen; antibacterial agents, such as amoxicillin; cholesteryl ester transfer protein (CETP) inhibitors, such as anacetrapib; anti-fungal agents, such as isoconazole; sepsis treatments, such as drotrecogin-α; steroidals, such as hydrocortisone; local or general anesthetics, such as ketamine; norepinephrine reuptake inhibitors (NRIs) such as atomoxetine; dopamine reuptake inhibitors (DARis), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRis), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor Vila Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, btuetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors; (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin; olanzapine; clonidine; and guanfacine.

Combination therapy is intended to embrace administration of these therapies in a sequential manner, that is, wherein the rapidly infusing composition and one or more other therapies are administered at a different time, as well as administration of these therapies, or at least two of the therapies, in a substantially simultaneous manner. Substantially simultaneous administration can be accomplished, for example, by administering to the subject multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, the rapidly infusing composition formulated with CBD may be administered via buccal administration while a second therapeutic agent of the combination may be administered intravenously.

Combination therapy also can embrace the administration of the rapidly infusing composition in further combination with other biologically active ingredients and non-drug therapies. Examples of non-drug therapies may include, but are not limited to, deep brain stimulation, vagus nerve stimulation, transcranial magnetic stimulation, and proton beam therapy. Where the combination therapy further comprises a non-drug treatment, the non-drug treatment may be conducted at any suitable time so long as a beneficial effect from the co-action of the combination of the therapeutic agent(s) and non-drug treatment is achieved. For example, in appropriate cases, the beneficial effect is still achieved when the non-drug treatment is temporally removed from the administration of the therapeutic agents, perhaps by days or even weeks.

The examples below are intended to further illustrate the materials and methods of the present disclosure, and are not intended to limit the scope of the claims.

Where a numerical limit or range is stated herein, the endpoints are included. Also, all values and subranges within a numerical limit or range are specifically included as if explicitly written out.

As used herein the words “a” and “an” and the like carry the meaning of “one or more.”

The present disclosure also contemplates other embodiments “comprising”, “consisting of” and “consisting essentially of”, the embodiments or elements presented herein, whether explicitly set forth or not.

All patents and other references mentioned above are incorporated in full herein by this reference, the same as if set forth at length.

Obviously, numerous modifications and variations of the present invention are possible in light of the above teachings. It is therefore to be understood that, within the scope of the appended claims, the invention may be practiced otherwise than as specifically described herein.

### Examples

### Rapidly Infusing Composition

### Ingredients

The ingredients that were used to make the rapidly infusing composition and the placebo are given in Table 1. USP=United States Pharmacopeia. EP=European Pharmacopoeia. NF=National Formulary.

TABLE 1
Ingredients

| Ingredient | Primary Function | Specification |
|---|---|---|
| Gelatin | Matrix former | USP/EP/NF |
| Mannitol | Bulking agent | USP/EP |
| Lemon-lime flavor powder | Flavorant | Non-compendial |
| CBD isolate | ATI | Non-compendial |
| Sucralose | Sweetener | USP/NF |
| Acesulfame-K | Sweetener | USP/NF |
| FD&C Yellow #5 | Colorant | Non-compendial |
| Purified water | Vehicle | USP/EP |

An example rapidly infusing composition was made using the formulation given in Table 2. The amount of each component is expressed in terms of weight percentage relative to a total weight (100%). The weight percentage of each component in the drug product suspension is on a wet basis (prior to removal of water). The weight percentage of each component in the rapidly infusing composition is on a dry basis (after removal of water).

TABLE 2
Example rapidly infusing composition formulation

| | Drug product | | | |
|---|---|---|---|---|
| | suspension | Rapidly Infusing Composition | | |
| | % wt./wt. | wt./unit | | % wt./wt. |
| Ingredient | (wet) | (dry) | | (dry) |
| Gelatin | 3.5 | 10.5 | mg | 22.7 |
| Mannitol | 3.0 | 9 | mg | 19.4 |
| Lemon-lime flavor | 0.2 | 0.6 | mg | 1.3 |
| powder | | | | |
| CBD isolate | 8.4 | 25 | mg | 54.0 |
| Sucralose | 0.2 | 0.6 | mg | 1.3 |
| Acesulfame-K | 0.2 | 0.6 | mg | 1.3 |

| | | | |
|---|---|---|---|
| FD&C Yellow #5 | Trace | Trace | Trace |
| Purified water | 84.5 | Removed during | Removed during |
| | | manufacture | manufacture |
| Total | 100.0 | — | 100.0 |

Methods of making the rapidly infusing composition
Purified water was charged to a pot and mixed using an overhead stirrer as an agitating device.
With agitation, the requisite amount of gelatin and mannitol were dispersed, and the mixture was heated until the excipients were dissolved.
Once dissolved, the sweeteners sucralose and acesulfame-K were added and allowed to dissolve.
The solution was cooled to 30° C., moved to an overhead homogenizer, and then the requisite amount of cannabidiol (CBD) isolate was charged and dispersed using the homogenizer to micronize the CBD and create a drug product suspension.
The requisite amount of Lemon-Lime flavor was charged and mixed for 10 minutes, then the FD&C Yellow #5 colorant was added.
The resulting drug product suspension was transferred to a second overhead mixer and maintained at a temperature of 30° C. for the ensuing dosing operation.
In a blistering machine equipped with a dosing system, blister pockets were filled with a target dose weight of 300.0 mg of the drug product suspension.
The product was frozen in a suitable cryochamber and then the blister trays were transferred from the cryochamber to a suitable refrigerated storage cabinet (temperature below 0° C.) prior to lyophilizing to keep the product frozen.
The frozen blisters were loaded from the refrigerated storage cabinet into lyophilizers and the product was lyophilized (water was sublimated) to form the rapidly infusing compositions.
When the lyophilizing cycle was completed, the rapidly infusing compositions were transferred from the lyophilizers to the blistering machine where the blister trays were heat sealed with lidding material.
The resulting tablets are flat-topped circular units approximately 15 mm in diameter with a convex bottom packaged in individual blister units (see also U.S. Provisional Application 63/114,181—incorporated herein by reference in its entirety).
The following tests were performed:
   A seal integrity test was performed at −0.5 Bar for 30 seconds, 1-minute soak time
   Visual inspection was performed
   Dry weight testing was performed

### Placebo

A placebo product was also formulated in the same manner as the rapidly infusing composition, with the exception that the placebo product was formulated without CBD.

### Clinical Trial Results

Final results from a clinical trial addressing post-surgical pain following shoulder arthroscopy, the methodology for which is described in U.S. patent application Ser. No. 17/225,738 filed Apr. 8, 2021, which is incorporated herein by reference, show that the rapidly infusing dosage form of the instant invention was shown to be safe and effective in reducing pain and improving patient satisfaction in the immediate per-operative period following arthroscopic rotator cup repair (ARCR).

On the safety issue, there were no serious adverse events reported among the participants in the experimental cohort, and only five (5) mild adverse events in total that could not confidently be attributed to CBD. This extremely low rate of adverse events, 9.4%, is basically unheard of in CBD trials and will likely make a tremendous difference in adherence to the therapy by future patients.

By contrast, clinical trial data for Epidiolex®, the only currently FDA-approved drug containing CBD, showed a far higher incidence of adverse events. For example, in epilepsy-related trials, adverse event rates varied from 45% (NCT02224703 at “mid dose” of 20 mg/kg/day) to 94% (NCT02564952 at 20-30 mg/kg/day) and a Parkinson's disease related trial (NCT02818777) experienced adverse events in 100% of the subjects. Results for pain-related clinical trials of Sativex®, an oromucosal spray containing both THC and CBD (which is not approved for any indication in the United States), also showed far more adverse events, with serious adverse events occurring in as high as 45.6% of patients (NCT01337089) and other adverse events (not including serious adverse events) occurring in as high as 97% of patients (NCT01606176) in certain trials.

1. A method for the treatment of Tourette syndrome in a subject, the method comprising:
administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD).

2. The method of claim 1, wherein the rapidly infusing composition is lyophilized.

3. The method of claim 1, wherein the rapidly infusing composition has a disintegration time of approximately 1 to 30 seconds in deionized water maintained at 37° C.±2° C.

4. The method of claim 1, wherein the rapidly infusing composition has a disintegration time of approximately 1 to 5 seconds in deionized water maintained at 37° C.±2° C.

5. The method of claim 1, wherein the gelatin is present in the rapidly infusing composition in an amount of 10 to 35 wt. %, based on a total weight of the rapidly infusing composition on a dry basis.

6. The method of claim 1, wherein the gelatin is bovine gelatin.

7. The method of claim 1, wherein the sugar alcohol is present in the rapidly infusing composition in an amount of 5 to 35 wt. %, based on a total weight of the rapidly infusing composition on a dry basis.

8. The method of claim 1, wherein the sugar alcohol comprises mannitol.

9. The method of claim 1, wherein the CBD is present in the rapidly infusing composition in an amount of 20 to 70 wt. %, based on a total weight of the rapidly infusing composition on a dry basis.

10. The method of claim 1, wherein the rapidly infusing composition is formulated with a solid form of the CBD.

11. The method of claim 1, wherein the rapidly infusing composition is formulated with a solid form of the CBD having a purity between 95 and 99.9 wt. %.

12. The method of claim 1, wherein the rapidly infusing composition is formulated with a solid form of the CBD that has been micronized to have a D50 diameter between 1 and 50 μm.

13. The method of claim 1, wherein the rapidly infusing composition further comprises at least one selected from the group consisting of a sweetener, a flavorant, and a colorant.

14. The method of claim 13, wherein the rapidly infusing composition comprises the flavorant, and the flavorant comprises lemon-lime flavor.

15. The method of claim 13, wherein the rapidly infusing composition comprises the colorant, and the colorant comprises FD&C Yellow #5.

16. The method of claim 13, wherein the rapidly infusing composition comprises the sweetener, and the sweetener comprises a mixture of sucralose and acesulfame-K.

17. The method of claim 1, wherein the rapidly infusing composition is administered to the subject via the buccal mucosa.

18. The method of claim 1, wherein the therapeutically effective amount of CBD is from 0.1 mg/kg/day to less than 7 mg/kg/day.

19. The method of claim 1, wherein the therapeutically effective amount of CBD is from 0.1 mg/kg/day to less than 5 mg/kg/day.

20. The method of claim 1, wherein the rapidly infusing composition is administered to the subject 1 to 3 times per day.

21. The method of claim 1, wherein CBD is the only active therapeutic ingredient in the rapidly infusing composition.

22. The method of claim 21, wherein the subject is not administered a cannabinoid other than CBD.

23. The method of claim 1, wherein the subject presents with at least one symptom of an abnormal involuntary movement selected from the group consisting of an eye movement, a facial movement, a shoulder movement, a head movement, a respiratory movement, and a vocalization.

24. The method of claim 1, wherein the subject presents with a complex symptom.

25. The method of claim 24, wherein the complex symptom is at least one speech-related symptom selected from the group consisting of coprolalia, echolalia, and palilalia.

26. The method of claim 24, wherein the complex symptom is a motor-related symptom involving several muscle groups.

27. The method of claim 26, wherein the complex symptom is at least one selected from the group consisting of hopping, jumping, bending, twisting, brushing hair, throwing objects, touching objects, copropraxia, and echopraxia.

28. The method of claim 23, wherein the subject presents with at least two symptoms selected from the group, and
wherein the at least two symptoms are exhibited simultaneously as a complex symptom.

29. The method of claim 1, wherein the subject presents with at least one condition selected from the group consisting of stuttering, attention deficit hyperactivity disorder, obsessive-compulsive disorder, and autism spectrum disorder.

30. The method of claim 1, wherein the subject is administered the rapidly infusing composition in the absence of an environmental modification comprising a trigger.

31. The method of claim 1, wherein a tic severity or frequency in the subject is reduced by at least 10%, relative to a severity or frequency observed prior to administration of the rapidly infusing composition.

32. The method of claim 1, wherein a tic severity or frequency in the subject is reduced by at least 20%, relative to a severity or frequency observed prior to administration of the rapidly infusing composition.

33. The method of claim 1, wherein a tic severity or frequency in the subject is reduced by at least 40%, relative to a severity or frequency observed prior to administration of the rapidly infusing composition.

34. The method of claim 1, wherein a premonitory urge for tics of the subject as measured by a Premonitory Urge for Tics Scale is reduced by at least 20%, relative to a premonitory urge for tics prior to administration of the rapidly infusing composition.

35. The method of claim 1, wherein a tic severity of the subject as measured by the Yale Global Tic Severity Scale is reduced by at least 10%, relative to a tic severity prior to administration of the rapidly infusing composition.

36. The method of claim 35, wherein the tic severity of the subject as measured by the Yale Global Tic Severity Scale is reduced by at least 25%, relative to the tic severity prior to administration of the rapidly infusing composition.

37. The method of claim 36, wherein the tic severity of the subject as measured by the Yale Global Tic Severity Scale is reduced by at least 50%, relative to the tic severity prior to administration of the rapidly infusing composition.

38. The method of claim 1, wherein the occurrence of tics in the subject is reduced completely.

39. The method of claim 1, wherein the subject is additionally treated with deep brain stimulation.

40. The method of claim 1, wherein the rapidly infusing composition is administered in combination with at least one drug selected from the group consisting of a dopamine precursor, a DOPA decarboxylase inhibitor, a catechol-O-methyl transferase (COMT) inhibitors, a dopamine receptor agonist, a neuroprotective agent, an NMDA antagonist, an anti-psychotic, a benzodiazepine, and a CYP2D6 inhibitor.

41. The method of claim 1, wherein the rapidly infusing composition is administered in combination with at least one anti-psychotic drug selected from the group consisting of chlorpromazine, levomepromazine, promazine, acepromazine, triflupromazine, cyamemazine, chlorproethazine, dixyrazine, fluphenazine, perphenazine, prochlorperazine, thiopropazate, trifluoperazine, acetophenazine, thioproperazine, butaperazine, perazine, periciazine, thioridazine, mesoridazine, pipotiazine, haloperidol, pimozide, fluphenazine, trifluperidol, melperone, moperone, pipamperone, bromperidol, benperidol, droperidol, fluanisone, oxypertine, molindone, sertindole, ziprasidone, flupentixol, clopenthixol, chlorprothixene, thiothixene, zuclopenthixol, fluspirilene, penfluridol, loxapine, clozapine, olanzapine, quetiapine, tetrabenazine, sulpiride, sultopride, tiapride, remoxipride, amisulpride, veralipride, levosulpiride, lithium, prothipendyl, risperidone, clotiapine, mosapramine, zotepine, pripiprazole, and paliperidone.

42. The method of claim 1, wherein the rapidly infusing composition is administered in combination with at least one benzodiazepine selected from the group consisting of alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

43. The method of claim 1, wherein the rapidly infusing composition is administered in combination with at least one CYP2D6 inhibitor selected from the group consisting of fluoxetine, paroxetine, bupropion, quinidine, cinacalcet, and ritonavir.

44. A method for the treatment of a tic disorder in a subject, the method comprising:
administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD),
wherein the subject does not have Tourette syndrome.

45. The method of claim 44, wherein the subject has at least one tic disorder selected from the group consisting of a pediatric autoimmune disorder associated with streptococcal infection (PANDAS), a transient tic disorder, and a chronic tic disorder.

46. The method of claim 44, wherein the subject has a movement disorder.
